SEQUENCE LISTING (4 sequences)

SEQ ID NO: 1 - protein, 331 aa, Unknown
  source: /note=“Description of Unknown Polypeptide Q2 sequence”
MKGFIAACLALAGLALGAPEVKRQKSDLTPQLLQNFHLFAQWSEAAGCNNQKAAGQPVTCDPGQCSLFDKHNATVVASFIGDVLDTRGFVGVDHVDKLIVVSFRGTTSVRNWIADLLFFQVSCDLTPGCLAHAGFYASWEEVKSRVLDAVKKAKAANPSYRVVATGYSLGGAIATLAVAYIRQQGHAADLYTYGGPRVGNLALAEFITNQPGGEYRITHKDDPVPRLPPLILNYRHTSPEYWIDPGNDRTVSLDEVQYCPGYSNTDCNGGTDGLNLDSHGFYFQSFIGCGPGSTPWRRASDISDKELEDRVNLFAELDRQLAKSLHEAGEQ

SEQ ID NO: 2 - DNA, 996 bp, Unknown
  source: /note=“Description of Unknown Polypeptide Q2 encoding sequence”
atgaagggtttcatagccgcttgcctcgccctggcgggcctagccctgggagcccccgaggtcaagaggcagaagagcgacctcaccccgcagctgctgcaaaacttccatctgttcgcccagtggtccgaggcggctgggtgcaacaaccaaaaggcagccgggcagcctgtgacctgcgacccgggccagtgctctctgttcgacaagcacaacgccaccgtcgtcgcctccttcatcggtgacgtgctcgacactcggggtttcgtcggcgttgaccatgtggataagctgattgtcgtctccttccggggtaccacctcggtgcggaactggatcgccgacctcctcttcttccaagtgtcctgcgacctgactcccggctgcctcgcccacgcgggcttctacgcgtcgtgggaggaggtcaagtcgcgcgtgctcgacgccgtgaagaaggccaaggcggccaacccgagctaccgcgtcgtggcgacgggctactcgctgggcggcgccatcgccaccttggccgtcgcctacatccgccagcaagggcacgccgccgacctgtacacgtacggcggcccgcgcgtcggcaacctcgccctggccgagttcatcacgaaccagcccggcggcgagtaccgcatcacgcacaaggacgaccccgtcccgcgcctcccgcccttgatcctcaactaccgccacacgagccccgagtactggatcgacccgggcaacgaccgcaccgtctccctcgacgaggtccagtactgcccgggctactccaacacggactgcaacggcggcaccgacggcctcaacctcgactcgcacggcttctacttccagagcttcatcggctgcggccccggcagcaccccgtggcgccgcgccagcgatatcagcgacaaggagctcgaggacagggtcaacttgtttgccgagctcgacaggcagctggccaagagcctgcacgaggccggcgagcagtga

SEQ ID NO: 3 - DNA, 993 bp, Artificial Sequence
  source: /note=“Description of Artificial Sequence Synthetic polynucleotide”
atgaaaggattcattgctgcgtgccttgctttagcgggacttgcgcttggagcgcccgaggttaaacgtcaaaagagtgatctgacaccccagcttcttcaaaattttcatttatttgcccagtggagcgaagcggcggggtgcaataaccagaaagccgcggggcaacctgtgacatgtgaccccggacagtgttcactgttcgataagcacaacgcaacggtcgtggcgagtttcatcggggacgtgttggacactcgtggatttgtaggggtagaccacgtagataagctgattgtcgtaagctttcgcggaacaacatccgttcgtaattggatcgctgacctgcttttctttcaggtaagttgtgaccttacaccaggctgtttggcccatgccgggttttacgcaagctgggaggaggtcaaatcgcgcgttttagacgcagtcaaaaaggcaaaagcagcgaacccttcataccgcgtagttgctactggatatagtcttggcggtgcaatcgctacgttagccgttgcctatattcgccagcaaggccatgccgcggatctttatacctacggtggtccgcgcgttggaaacctggcattggccgaattcattactaatcagcccggcggggagtatcgcattacgcacaaagatgatcctgtgccccgccttccacctttaattctgaattatcgccacacgtccccagaatattggattgacccgggtaacgaccgcaccgtctctttagacgaagtccagtactgtccgggatactcgaataccgactgcaatggaggtactgacggattaaatttagatagtcacggattctattttcagtcttttatcggctgtggaccagggtcgactccatggcgtcgtgcatcagatatttcggacaaggagctggaagaccgtgtcaatttgttcgcggagctggaccgtcaacttgcgaagtcccttcacgaagcgggggagcaa

SEQ ID NO: 4 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2023/0072496 A1
(43) Pub. Date: Mar. 9, 2023

(54) POLYPEPTIDE COMPOSITIONS AND USES THEREOF

(71) Applicant: LIBERTY BIOSECURITY, LLC, Arlington, VA (US)

(72) Inventors: Keith BALLARD, Worcester, MA (US); Samuel JORDAN, Worcester, MA (US); Laura VON DER PORTEN, Worcester, MA (US); Timothy AVERY, Worcester, MA (US); Kyle LANDRY, Worcester, MA (US)

(21) Appl. No.: 17/917,799

(22) PCT Filed: Apr. 9, 2021
(86) PCT No.: PCT/US2021/026699

§ 371 Date: Oct. 7, 2022

Related U.S. Application Data
(60) Provisional application No. 63/008,194, filed on Apr. 10, 2020.

(51) Int. Cl. A01N 63/50; C07K 14/00; A61K 45/06; A01P 1/00; C12N 9/18; C12N 15/64

(52) U.S. Cl. CPC A01N 63/50; C07K 14/00; A61K 45/06; A01P 1/00; C12N 9/18; C12Y 301/01074; C12N 15/64; A61K 38/00

(57) ABSTRACT

This technology relates in part to polypeptide compositions and to methods of treatment using the compositions, such as the treatment of diseases or conditions that are accompanied by microbial or viral infections, wounds or sores and the treatment of surfaces contaminated by microbes, viruses or other pathogens.

### CROSS REFERENCE TO RELATED APPLICATIONS

This application claims priority to U.S. Provisional Patent Application No. 63/008,194, filed on Apr. 10, 2020, and PCT International Application no. PCT/US2021/026699, filed on Apr. 9, 2021, both of which are incorporated herein by reference in its entirety.

### FIELD

The technology relates to polypeptide compositions for a variety of uses, including the treatment of diseases or conditions that are susceptible to and/or accompanied by microbial infections, tissue injury or damage, wounds or sores and the treatment of surfaces contaminated by microbes and other pathogens.

### BACKGROUND

The treatment of certain diseases or conditions that are susceptible to and/or accompanied by microbial infections, tissue injury or damage, wounds or sores in or on a subject can sometimes pose challenges due to resistance of the microbes to conventional treatments (e.g., with antibiotics, antiseptics and/or oxidizing biocides). Surfaces contaminated by microbes also can sometimes be resistant to disinfection. What is needed are stable compositions, including compositions that are thermally stable at higher temperatures e.g., under certain sterile conditions, in or on a living body, e.g., a homeothermic animal such as a human, or when inflammation is present, which either, kill, inhibit the growth of, or otherwise eliminate microbes, or which permit treatment/disinfection by overcoming, in whole or in part, resistance to traditional approaches that are used in such situations. Stable compositions are needed, for example, which either: (a) kill, inhibit the growth of, degrade, or otherwise reduce or eliminate microbes that are associated with infections, wounds or sores, or that are associated with certain surfaces such as in industrial, dental or health care settings, or (b) render such microbes susceptible to agents that can kill them and/or inhibit their growth and/or degrade, reduce the number of, and/or eliminate them, e.g., from a surface.

### SUMMARY

Provided herein in certain aspects is a polypeptide containing a consecutive sequence of amino acids that is 80% or more identical, or 90% or more identical to a corresponding consecutive sequence of amino acids set forth in SEQ ID NO:1 (which is referred to herein as “Polypeptide Q2”). In some embodiments, the sequence of amino acids is x80% or more identical, or 90% or more identical to the sequence of amino acids set forth in SEQ ID NO: 1, and in some embodiments, the polypeptide is a polypeptide of SEQ ID NO. 1. In some embodiments, the polypeptide exhibits an enzyme activity and/or biofilm removal activity. In some embodiments, the enzyme activity includes a hydrolase activity, such as, for example, a hydrolase activity that acts on carboxylic ester bonds, e.g., an esterase, such as, for example, a serine esterase. In some aspects, the enzyme activity includes a cutinase activity. Also provided herein is a polypeptide that includes a portion of a sequence of amino acids that is 80% or more identical, 90% or more identical, or 100% identical to the sequence of amino acids set forth in SEQ ID NO: 1, wherein the portion exhibits enzyme activity and/or biofilm removal activity. In some embodiments, the enzyme activity includes a hydrolase activity, such as, for example, a hydrolase activity that acts on carboxylic ester bonds, e.g., an esterase, such as, for example, a serine esterase. In some aspects, the enzyme activity includes a cutinase activity. In some embodiments of the polypeptides provided herein, the polypeptide is an isolated, recombinant, or synthetically produced polypeptide.

Also provided herein in certain aspects are compositions, e.g., polypeptide compositions, that include one or more polypeptides selected from among the polypeptide of SEQ ID NO:1, and polypeptides that include a consecutive sequence of amino acids that is 80% or more, or 90% or more identical to a corresponding consecutive sequence of amino acids of Polypeptide Q2. Included among the compositions provided herein are pharmaceutical compositions, e.g., for the treatment of diseases or conditions that are susceptible to and/or accompanied by infections, tissue injury or damage, wounds, sores or burns in or on a subject, compositions for treating, e.g., disinfecting, a surface, e.g., in industrial, dental or health care settings, and compositions for removing a biofilm. Also provided herein are combinations of one or more polypeptides provided herein and one or more agents, such as a therapeutically active agent (e.g., a therapeutically active agent that does not include a polypeptide provided herein) and/or a surface treatment agent (e.g., a surface treatment agent that does not include a polypeptide provided herein). In one aspect, a combination provided herein comprises one or more, such as, e.g., two, separate agents (for example a polypeptide provided herein and a separate therapeutically active agent or surface treatment agent) for combined treatment, e.g., treatment of a subject or treatment of a surface. Such agents of a combination are administered or applied as separate substances but for combined use. For example, such separate agents may be administered serially, sequentially or concurrently but as separate agents that are not mixed prior to administration. In some aspects, the agents of a combination may be mixed together and administered as a single composition.

In certain aspects, a polypeptide, composition or combination provided herein is thermally stable. In aspects, one or more polypeptides of the compositions or combinations provided herein are thermally stable. The term “thermally stable,” as used herein, refers t retains at least about 20%, 25%, 30%, 35%, 40%, 45%, 50%, 55%, 60%, 65%, 70%, 75%, 80%, 81%, 82% , 83%, 84%, 85%, 86%, 87%, 88%, 89%, 90%, 91%, 92%, 93%, 94%, 95%, 96%, 97%, 98%, 99% or more, up to 100of its activity, e.g., for the treatment of a subject and/or surface, for treating or disinfecting surfaces, and/or for removing biofilm, after experiencing elevated temperatures greater than or about 30° C.,such as 30° C. to 125° C. or more, generally about 30° C. to about 37° C., 38° C., 39° C., 40° C., 41° C., 42° C., 43° C., 44° C. or 45° C. or greater, or about 40° C. to about 120° C. or about 45° C. to about 125° C., or about 37° C., 38° C., 39° C., 40° C., 41° C., 42° C., 43° C., 44° C. or 45° C. or more.

The polypeptides provided herein and/or used in the compositions provided herein can be isolated and/or otherwise obtained from a number of microbes, such as bacteria and fungi, or can be obtained recombinantly or through synthetic production processes. In certain aspects, the polypeptides provided herein and/or used in the compositions provided herein can be obtained from thermophilic microbes, i.e., microbes that can survive at high temperatures greater than or about 30° C., such as 3020 C. to 125° C. or more, generally about 30to about 37° C., 38° C., 39° C., 40° C., 41° C., 42° C., 43° C., 44° C. or 45° C., or greater, or about 40° C. to about 120° C. or about 45° C. to about 125° C., or about 37° C., 38° C., 39° C., 40° C., 41° C., 42° C., 43° C., 44° C. or 45° C., and/or contain polypeptides that are stable and/or active at high temperatures greater than or about 30° C., such as 30° C. to 125° C. or more, generally about 30° C. to about 37° C., 38° C., 39° C., 40° C., 41° C., 42° C., 43° C., 44° C. or 45° C. or greater, or about 40° C. to about 120° C. or about 45° C. to about 125° C., or about 37° C., 38° C., 39° C., 40° C., 41° C., 42° C., 3° C., 44° C. or 45° C. For example, the Polypeptide Q2 peptide provided herein and in compositions provided herein can be obtained from a thermophilic fungus, TM-417 (see, e.g., Salar, R. K., Thermophilic Fungi: Basic Concepts and Biotechnological Applications, CRC Press (2018), p. 282; submitted to the Index Fungorum as Parvabulbium thermostercus (Index Fungorum, Landry et al, 2021)).

In some aspects, the compositions provided herein, for example, pharmaceutical compositions, can be used to treat a disease or condition in a subject. In some aspects, the disease or condition is one that is susceptible to, associated with and/or accompanied by infections, tissues, such as animal (e.g., human) tissue, and/or tissue (e.g., surface tissue) injury or damage, including, for example, wounds, sores or burns in or on a subject. In some aspects, the disease or condition is accompanied by or associated with tissue, including, for example, epithelial tissue, connective tissue, or tissue membranes (e.g., mucous membranes, cutaneous membranes and serous membranes). Examples of a disease and/or condition associated with mucosa include, but are not limited to, an ulcer in the mucosal lining, such as a peptic ulcer, an infection of the lung mucosa in diseases such as cystic fibrosis or tuberculosis, infection of the gums and/or periodontium, such as in gingivitas, infection of the mouth and/or throat (e.g., strep throat and thrush, vaginitis and associated bacterial infections) or an infection in nasal passages, such as COVID-19. Examples of a disease and/or condition associated with an epithelial tissue membrane, e.g. a cutaneous membrane such as skin, include, but are not limited to, bacterial infections (e.g., cellulitis, impetigo), viral (e.g., herpes virus, human papillomavirus) infections (e.g., shingles, cold sores, and warts, and associated bacterial infections), and fungal infections (e.g., athlete's foot, ringworm and associated bacterial infections). Examples of tissue (e.g., surface tissue) injury or damage, include, but are not limited to, a wound, burn or a sore. The term “wound,” as used herein, refers to an injury, such as a cut, stab or tear, to an external or internal part of the body. In certain aspects, the wound can become infected, or can be associated with an infection. The term “sore,” as used herein, refers to an infected, inflamed and/or diseased area (e.g., a blister, lesion), generally appearing as a break in the skin or mucous membrane, e.g., the lining of an organ, such as the stomach. The term “ulcer” generally refers to a type of sore that erodes the mucous membrane or skin, generally as the result of a disease or other abnormal condition. The term “burn” generally refers to an injury to a tissue resulting from exposure to heat, friction, radiation, electricity or chemicals. Any of the diseases and conditions treated using compositions (e.g., pharmaceutical compositions) provided herein can be characterized by or associated with an infection. The term “infection,” as used herein, refers to a disease or condition, or a state associated with a disease or condition, in which one or more pathogenic agents such as bacteria, fungi (e.g., yeasts) or viruses, including, e.g., herpes viruses, human papillomavirus and coronaviruses, become established in or on the body of a subject.

A “subject,” as used herein, can be an animal, such as a human being. As used herein, “treating,” with reference to treating a subject with a disease or condition, means that the disease or condition, or symptoms associated with the disease or condition, are partially or totally alleviated, or the disease or condition and/or symptoms thereof remain static (do not progress) following treatment. Hence, treatment of a disease or condition encompasses prophylaxis, therapy and/or cure. Prophylaxis refers to prevention of a potential disease and/or a prevention of worsening of symptoms or progression of a disease. As used herein, “treatment” means any manner, e.g., by administration of a composition, e.g., a pharmaceutical composition, provided herein and, optionally, an additional therapeutically active agent and/or surface treatment agent, by which a condition, disorder or disease, or symptoms thereof, are ameliorated.

Thus, in certain aspects, provided herein is a pharmaceutical composition that includes: (a) one or more polypeptides containing the sequence of amino acids set forth in SEQ ID NO:1 (Polypeptide Q2) and/or containing a consecutive sequence of amino acids that is 80% or more, or 90% or more identical to a corresponding consecutive sequence of amino acids set forth in SEQ ID NO:1; and (b) a pharmaceutically acceptable excipient. Also provided, in some aspects, is a pharmaceutical composition for removing biofilm in or on a subject, which includes: (a) one or more polypeptides exhibiting biofilm removal activity and selected from among (i) a polypeptide of SEQ ID NO:1, (ii) a portion of the polypeptide of SEQ ID NO:1, wherein the portion exhibits biofilm removal activity, and/or (iii) a polypeptide that includes a consecutive sequence of amino acids that is 80% or 30more, or 90% or more identical to a consecutive sequence of amino acids set forth in SEQ ID NO:1 and exhibits biofilm removal activity; and (b) a pharmaceutically acceptable excipient.

In certain aspects, compositions provided herein, such as, for example, pharmaceutical compositions, compositions for treating a surface and/or removing biofilm, provided herein, and combinations provided herein, exhibit one or more of biofilm removal activity, microbicidal activity, antiviral, antifungal and microbiostatic activity. In aspects, compositions provided herein, such as, for example, pharmaceutical compositions or compositions for treating a surface and/or removing biofilm, provided herein, and combinations provided herein, exhibit biofilm removal activity and, optionally, one or both of microbicidal activity and microbiostatic activity. In certain aspects, a compositions provided herein, such as, for example, a pharmaceutical composition or composition for treating a surface and/or removing biofilm, provided herein, and a combination provided herein, does not exhibit one or both of microbicidal activity and microbiostatic activity.

The term “microbicidal” refers to the killing of microorganisms or microbes, used interchangeably herein. A microorganism, referred to interchangeably herein as a “microbial cell,” “microbe,” or “microbial organism,” is a microscopic organism that is multicellular or unicellular. Unicellular microorganisms may exist as a single cell or within a colony of cells. Many microorganisms are capable of dividing and proliferating. Microorganisms include prokaryotic microorganisms (e.g., bacteria), non-prokaryotic (e.g., eukaryotic) microorganisms and viruses. Examples of eukaryotic organisms include yeast, filamentous fungi, protists, plants, algae and amoeba. An organism or microorganism can include one or more of the following features: aerobe, anaerobe, filamentous, non-filamentous, monoploid, haploid, diploid, auxotrophic and/or non-auxotrophic, e.g., prototrophic. Some microorganisms are pathogenic and may be associated with and/or capable of causing disease. Pathogenic microorganisms, also 0ferred to as pathogens, include primary pathogens and opportunistic pathogens.

In certain aspects, the microbes are bacteria and the compositions provided herein, such as, for example, pharmaceutical compositions or compositions for treating a surface and/or removing biofilm, provided herein, and combinations provided herein, exhibit “bactericidal” activity, i.e., the ability to kill bacteria. In certain aspects, the microbes are fungi and the compositions provided herein, such as, for example, pharmaceutical compositions or compositions for treating a surface and/or removing biofilm, provided herein, and combinations provided herein, exhibit “fungicidal” activity, i.e., the ability to kill fungi. In certain aspects, the microbes are viruses and the compositions provided herein, such as, for example, pharmaceutical compositions or compositions for treating a surface and/or removing biofilm, provided herein, and combinations provided herein, exhibit “viricidal” activity, i.e., the ability to destroy or inactivate viruses. In aspects, compositions provided herein, such as, for example, pharmaceutical compositions or compositions for treating a surface, provided herein, and combinations provided herein, exhibit biofilm removal activity and, optionally, viricidal activity. In certain aspects, a composition provided herein, such as, for example, a pharmaceutical composition or composition for treating a surface and/or removing biofilm, provided herein, and a combination provided herein, does not exhibit viricidal activity.

The term “microbiostatic” refers to reducing, slowing or stopping the growth of microbes; the microbes eventually die, due to cessation of replication. In certain aspects, the microbes are bacteria and the compositions provided herein, such as, for example, pharmaceutical compositions or compositions for treating a surface and/or removing biofilm, provided herein, and combinations provided herein, exhibit “bacteriostatic” activity, i.e., the ability to reduce, slow or stop the growth of bacteria. In aspects, compositions provided herein, such as, for example, pharmaceutical compositions or compositions for treating a surface, provided herein, and combinations provided herein, exhibit biofilm removal activity and, optionally, one or both of bactericidal activity and bacteriostatic activity. In certain aspects, a composition provided herein, such as, for example, a pharmaceutical composition or composition for treating a surface and/or removing biofilm, provided herein, and a combination provided herein, does not exhibit one or both of bactericidal activity and bacteriostatic activity.

The term “fungistatic” refers to reducing, slowing or stopping the growth of fungi; the microbes eventually die, due to cessation of replication. In certain aspects, the microbes are fungi and the compositions provided herein, such as, for example, pharmaceutical compositions or compositions for treating a surface and/or removing biofilm, provided herein, and combinations provided herein, exhibit “fungistatic” activity, i.e., the ability to reduce, slow or stop the growth of fungi. In aspects, compositions provided herein, such as, for example, pharmaceutical compositions or compositions for treating a surface, provided herein, and combinations provided herein, exhibit biofilm removal activity and, optionally, one or both of fungicidal activity and fungistatic activity. In certain aspects, a composition provided herein, such as, for example, a pharmaceutical composition or composition for treating a surface and/or removing biofilm, provided herein, and a combination provided herein, does not exhibit one or both of fungicidal activity and fungistatic activity.

The term “biofilm” is a term of art that refers to a collective of one or more microorganisms, such as, for example, bacteria and fungi, that accumulate naturally on a variety of surfaces. Non-limiting examples of surfaces can include a surface of an inanimate object, e.g., household and industrial pipes, biomaterials such as contact lenses, medical devices including implants and urinary catheters, as well as a biological surface, e.g., plant and animal tissues. On such surfaces, microorganisms start to form a monolayer and produce an extracellular matrix or “slime” for protection. The matrix contains a variety of components including, but not limited to, extracellular polysaccharides, structural proteins, cell debris and nucleic acids; referred to as extracellular polymeric substances (EPS).

The close proximity of microorganisms to each other enables substrate exchange, the distribution of metabolic products and removal of toxic end products so that the different species can support each other. In addition, the structure of the biofilm communities can protect the microbes within them from attack by antimicrobials (e.g., antibiotics, when the microbes are bacteria), shear forces and the immune system. For example, when a disease or condition in a subject is characterized by or associated with a chronic wound, the formation of a biofilm, e.g., a bacterial biofilm, at the wound often can contribute to inability of the wound to heal by a variety of mechanisms including, but not limited to, the extracellular polysaccharide matrix acting as a physical barrier to host inflammatory cells, the inhibition of complement activation and other host defensive mechanisms leading to an ineffective inflammatory/immune response, the blockade and inactivation of antibiotics and other therapeutically active agents (such as antimicrobial agents), and antiseptic or disinfectant agents, and wound healing inhibition through direct inhibition of keratinocyte migration.

In certain aspects, the compositions provided herein, such as, for example, pharmaceutical compositions or compositions for treating a surface and/or removing biofilm provided herein, and combinations provided herein, contain polypeptide(s) that exhibit biofilm removal activity. “Biofilm removal activity,” as used herein, refers to activity that reduces and effects removal of a biofilm or portion thereof. Removal can be, for example, disruption, degradation, breakdown and/or destruction of biofilm that results in a reduction, e.g., in amount, of a biofilm (e.g., of an intact biofilm) and/or one or more components (e.g., extracellular matrix material, such as EPS, and/or cells, e.g., microbial cells, such as viable microbial cells) thereof. The reduction can be a partial reduction or complete reduction (e.g., elimination). Methods for quantitatively and qualitatively assessing the amount or extent of a biofilm are known in the art and/or described herein. In aspects, the polypeptide(s) exhibit biofilm removal activity and the polypeptide(s) is/are in an amount sufficient to remove at least 20% of the biofilm (or reduce the amount of biofilm by at least 20%) associated with a particular area, e.g., an area of a surface, a tissue (such as animal (e.g., human) tissue), e.g., mucosa, a site of tissue (e.g., surface tissue) injury or damage, a site of infection, a wound, a sore or a burn in or on a subject. In some aspects, the compositions provided herein, such as pharmaceutical compositions or compositions for treating a surface and/or removing biofilm provided herein, and combinations provided herein, the polypeptide(s), or both the composition (or combination) and the polypeptide(s) do not exhibit microbicidal activity or bactericidal activity.

The compositions provided herein, such as pharmaceutical compositions or compositions for treating a surface and/or removing biofilm provided herein, and combinations provided herein, can contain, in certain aspects, a single polypeptide, or can contain, in aspects, a plurality of polypeptides. In certain aspects, the compositions provided herein, such as pharmaceutical compositions or compositions for treating a surface and/or removing biofilm provided herein, and combinations provided herein, consist essentially of a single polypeptide. The phrase “consist(s) essentially of,” as used herein, means that components other than the recited components, if present, do not materially alter the activity of the recited components. Thus, for example, in the context of the compositions, e.g., pharmaceutical compositions or compositions for treating a surface and/or removing biofilm, provided herein, the compositions may include one or more components other than a single polypeptide having the aforementioned sequence characteristics and properties. In some aspects, the compositions, e.g., pharmaceutical compositions or compositions for treating a surface and/or removing biofilm, provided herein consist of a single polypeptide.

In certain aspects, at least one polypeptide in the compositions provided herein, such as pharmaceutical compositions or compositions for treating a surface and/or removing biofilm provided herein, and combinations provided herein, exhibits biofilm removal activity and in certain aspects, the compositions provided herein, such as pharmaceutical compositions or compositions for treating a surface and/or removing biofilm provided herein, and combinations provided herein, contain only one polypeptide exhibiting biofilm removal activity. In some aspects, the polypeptide exhibiting biofilm removal activity contains the sequence of amino acids set forth in SEQ ID NO:1 (Polypeptide Q2). In some aspects, the polypeptide exhibiting biofilm removal activity consists essentially of the sequence of amino acids set forth in SEQ ID NO:1 (Polypeptide Q2) and in some aspects, the polypeptide exhibiting biofilm removal activity consists of the sequence of amino acid set forth in SEQ ID NO:1 (Polypeptide Q2).

In certain aspects, the compositions provided herein, such as pharmaceutical compositions or compositions for treating a surface and/or removing biofilm provided herein, and combinations provided herein, that include at least one polypeptide having biofilm removal activity can be used to remove or reduce the amount of biofilm associated with an area of a surface, a tissue (such as animal (e.g., human) tissue), e.g., mucosa, a site of tissue (e.g., surface tissue) injury or damage, a site of infection, a wound, a sore or a burn in or on a subject. In some aspects, the biofilm is associated with the gums (i.e., gingiva) and/or periodontium, and in some aspects, the subject having biofilm associated with the gums and/or periodontium has gingivitis and/or periodontal disease. In some aspects, the biofilm is associated with a tooth and, in some aspects, the subject having biofilm associated with a tooth has tooth decay (dental caries). In some aspects, the biofilm is associated with a wound and, in aspects, the wound is a chronic wound. In certain aspects, the subject having biofilm associated with a wound has a disease or condition selected from among wound infection, an ischemic condition, a metabolic condition, immunosuppression and radiation. In some aspects, the subject has a metabolic condition and, in aspects, the metabolic condition is diabetes mellitus. In some aspects, the subject has an ischemic condition, and, in aspects, the ischemic condition is associated with atherosclerosis, peripheral vascular disease and/or venous insufficiency.

In certain aspects, the biofilm is associated with mucosa; in aspects the subject has cystic fibrosis or tuberculosis. In certain aspects, the subject has COVID-19. In certain aspects the subject has an infection (e.g., strep throat, thrush, vaginitis). In certain aspects the biofilm is associated with an epithelial tissue membrane, e.g., a cutaneous membrane such as skin; in aspects the subject has an infection (e.g., cellulitis, impetigo, shingles, cold sore, wart, athlete's foot, or ringworm).

The compositions provided herein, such as pharmaceutical compositions or compositions for treating a surface and/or removing biofilm provided herein, and combinations provided herein, can be in any form, including, but not limited to, a liquid, solid, semi-solid or mixture of a liquid, solid and/or semi-solid. For example, the compositions and combinations can be, or include, a solution, dispersion, suspension, emulsion, or colloid. The compositions provided herein, such as pharmaceutical compositions or compositions for treating a surface and/or removing biofilm provided herein, and combinations provided herein, can be formulated in a variety of ways including, but not limited to, a gel, ointment, cream, lotion, oil, butter, paste, balm, stick, foam, serum, mousse, patch, spray, aerosol, powder, or lyophile (or lyophilizate). The compositions provided herein, such as pharmaceutical compositions or compositions for treating a surface and/or removing biofilm provided herein, and combinations provided herein, can be formulated, in certain aspects, as a sustained release formulation. In certain aspects, the formulations provided herein can be formulated for single dosage administration. In some aspects, the formulations provided herein can be formulated for multiple dosage administration.

In certain aspects, the compositions provided herein, such as pharmaceutical compositions or compositions for treating a surface and/or removing biofilm provided herein, and combinations provided herein, include an additional therapeutically active agent. For example, the additional therapeutically active agent can be selected from among one or more of an analgesic agent, an anti-inflammatory agent and an antimicrobial agent (e.g., an antibacterial agent, an antifungal agent or an antiviral agent). In certain aspects, for example, if the composition, e.g., pharmaceutical composition, or combination is for treating a disease or condition associated with a bacterial infection, the additional therapeutically active agent can be an antibiotic. The antibiotics can be bacteriostatic, in certain aspects, such as, for example, tetracyclines, sulfonamides, spectinomycin, trimethoprim, chloramphenicol, macrolides and lincoamides. In certain aspects, the antibiotics can be bactericidal, such as, for example, the beta-lactam antibiotics, including, but not limited to, penicillin and derivatives thereof, cephalosporins, monobactams and carbapenems. Bactericidal antibiotics further include, but are not limited to, daptomycin, fluoroquinolones, metronidazole, nitrofurantoin, co-trimoxazole and telithromycin. In certain aspects, examples of antibiotic agents that can serve as additional therapeutically active agents in the compositions, e.g., pharmaceutical compositions, and combinations provided herein include, but are not limited to, Aminoglycosides; Amphenicols; Ansamycins; Carbacephems; Carbapenems; Cephalosporins or Cephems; Cephamycins; Clavams; Cyclic lipopeptides; Diaminopyrimidines; Ketolides; Lincosamides; Macrolides; Monobactams; Nitrofurans; Oxacephems; Oxazolidinones; Penems, thienamycins and miscellaneous beta-lactams; Penicillins; Polypeptides antibiotics; Quinolones; Sulfonamides; Sulfones; Tetracyclines; and other antibiotics (such as Clofoctols, Fusidic acids, Hexedines, Methenamines, Nitrofurantoins Nitroxolines, Ritipenems, Taurolidines, Xibomols). In certain aspects, the action of the additional therapeutically active agent is potentiated by the action of the at least one polypeptide described herein.

In some aspects, for example, if the composition, e.g., pharmaceutical composition, or combination is for treating cystic fibrosis or tuberculosis, the additional therapeutically active agent can be selected from among one or more of: an antibiotic for treating and preventing lung infections, examples of antibiotics being among those described above, an anti-inflammatory drug, a mucus-thinning drug, a bronchodilator, a pancreatic enzyme and drugs such as ivacaftor, lumacaftor, tezacaftor or combinations thereof for treating subjects with certain mutations in the cystic fibrosis transmembrane conductance regulator (CFTR) gene.

In some aspects, for example, if the composition, e.g., pharmaceutical composition, or combination is for treating an infection associated with shingles or a cold sore, the additional therapeutically active agent can be an antiviral agent such as, for example, a nucleoside analog (e.g., valacyclovir, acyclovir). In some aspects, for example, if the composition, e.g., pharmaceutical composition, or combination is for treating an infection associated with a mycotic disease (e.g., athlete's foot, mycotic nails or ringworm), the additional therapeutically active agent can be an antifungal (antimycotic) agent such as, for example, imidazole or derivative thereof (e.g., fluconazole, clotrimazole, ketoconazole, miconazole), or a hydroxypyridone (e.g., ciclopirox).

In certain aspects, the compositions, e.g., pharmaceutical compositions, provided herein and the additional therapeutically active agent can be co-formulated. In aspects, the compositions, e.g., pharmaceutical compositions, provided herein and the additional therapeutically active agent can be independently formulated.

Also provided herein are compositions for treating a surface and/or removing biofilm, wherein the compositions include one or more polypeptides selected from among (i) a polypeptide of SEQ ID NO:1, (ii) a portion of the polypeptide of SEQ ID NO:1, wherein the portion exhibits enzyme activity and/or biofilm removal activity, and/or (iii) a polypeptide that includes a consecutive sequence of amino acids that is 80% or more, or 90% or more identical to a consecutive sequence of amino acids set forth in SEQ ID NO:1. In some aspects, the compositions for treating a surface and/or removing biofilm includes one or more polypeptides exhibiting biofilm removal activity and/or enzyme activity selected from among (i) a polypeptide of SEQ ID NO:1, (ii) a portion of the polypeptide of SEQ ID NO:1, wherein the portion exhibits biofilm removal activity and/or enzyme activity, and/or (iii) a polypeptide that includes a consecutive sequence of amino acids that is 80% or more, or 90% or more identical to a consecutive sequence of amino acids set forth in SEQ ID NO:1 and exhibits biofilm removal activity or antiviral activity. Treating, with reference to a surface, refers to contacting a surface generally in the process of decontaminating the surface. Treating can include, for example, one or more of disinfection, antisepsis, sanitization and cleaning. The term “disinfection,” as used herein, includes biofilm removal activity, microbicidal (or bactericidal, fungicidal or viricidal) activity, microbiostatic (or bacteriostatic or fungistatic) activity, or any combination thereof. In certain aspects, the activity is antiviral activity or antifungal activity. The term “antisepsis,” as used herein, refers to disinfection of a biological surface, such as living tissue. The term “cleaning,” as used herein, refers to physical removal of contaminants away from a site of contamination and can be without microbicidal (or bactericidal, fungicidal or viricidal) activity and/or microbiostatic (or bacteriostatic or fungistatic) activity. The term “sanitization,” as used herein, refers to reducing the amount of a contaminant. The surface can be a hard surface, a soft surface or a porous surface. In certain aspects, the surface is a porous surface and the porous surface is selected from among skin, keratin and internal organs. Disinfection of a surface can include one or more of biofilm removal activity, bacteriostatic activity and bactericidal activity, antifungal, and antiviral activity. In certain aspects, the disinfection consists of biofilm removal activity. In aspects, the disinfection includes biofilm removal activity and one or more of bacteriostatic activity and bactericidal activity.

Provided herein are methods of treating a disease or conditions in a subject that includes administering, to a subject in need thereof, one or more polypeptides, isolated or otherwise obtained (e.g., recombinantly or synthetically produced), which are provided herein. In some aspects, the method includes administering, to a subject in need thereof, a therapeutically effective amount of one or more of the polypeptides provided herein. Also provided herein are methods of treating a disease or condition in a subject that includes administering, to a subject in need thereof, a composition provided herein, such as pharmaceutical composition, or composition for treating a surface (e.g, a biological surface) and/or removing biofilm provided herein, or combination provided herein. In some aspects, the methods include administering, to a subject in need thereof, a therapeutically effective amount of a composition provided herein, such as pharmaceutical composition, or composition for treating a surface (e.g, a biological surface) and/or removing biofilm provided herein, or combination provided herein. In some aspects, the disease or condition is one that is susceptible to, associated with and/or accompanied by infection, a tissue, such as animal (e.g., human) tissue, and/or tissue (e.g., surface tissue) injury or damage, including, for example, wounds, sores or burns in or on a subject. In some aspects, the disease or condition is accompanied by or associated with tissue, including, for example, epithelial tissue, connective tissue, or tissue membranes (e.g., mucous membranes, cutaneous membranes and serous membranes). In aspects, the subject has a disease or condition selected from among an infection, ulcers, a wound and/or wound infection, an ischemic condition, a metabolic condition, immunosuppression, radiation, tuberculosis, COVID-19, and cystic fibrosis. In some aspects, the disease or condition is a metabolic condition. In certain aspects, the metabolic condition is diabetes mellitus.

In certain aspects of the methods provided herein, there is biofilm associated with the disease or condition. In some aspects, the biofilm associated with the disease or condition is reduced by at least 20% upon treatment using the methods provided herein.

Also provided herein methods of treating a disease or condition in a subject that includes administering, to a subject in need thereof, one or more polypeptides, isolated or otherwise obtained, which are provided herein, or a composition provided herein, such as pharmaceutical composition, or composition for treating a surface (e.g, a biological surface) and/or removing biofilm provided herein, or combination provided herein, and an additional therapeutically active agent or surface treatment agent that is co-formulated with the polypeptide(s) in the composition or combination, or is independently formulated. In some aspects, the disease or condition is one that is susceptible to, associated with and/or accompanied by infection, a tissue, such as animal (e.g., human) tissue, and/or tissue (e.g., surface tissue) injury or damage, including, for example, wounds, sores or burns in or on a subject. In certain aspects, the disease or condition is associated with a wound, sore or infection. In aspects, the additional therapeutically active agent is selected from among one or more of: an analgesic agent, an anti-inflammatory agent and an antimicrobial agent (e.g., an antibacterial agent, an antifungal agent or an antiviral agent). In some aspects, the subject has a disease or condition selected from among an infection, wound and/or wound infection, an ischemic condition, a metabolic condition, immunosuppression, radiation, tuberculosis, COVID-19, and cystic fibrosis. In certain aspects, the disease or condition is a metabolic condition. In aspects, the metabolic condition is diabetes mellitus.

In certain aspects, the therapeutically active agent is an antimicrobial agent and, in some aspects, the antimicrobial agent is an antibiotic. In aspects of the methods, the pharmaceutical composition and the additional therapeutically active agent can be administered serially, sequentially, intermittently, concurrently or simultaneously. In some aspects, the pharmaceutical composition is administered prior to administering the therapeutically active agent.

In certain aspects of the methods provided herein for treatment by administering a polypeptide or a composition provided herein, such as pharmaceutical composition, or composition for treating a surface (e.g, a biological surface) and/or removing biofilm provided herein, or combination provided herein, and an additional therapeutically active agent, there is biofilm associated with the disease or condition. In some aspects, the biofilm associated with the disease or condition is reduced by at least 20%.

Also provided herein are devices that include a polypeptide or a composition provided herein, such as pharmaceutical composition, or composition for treating a surface (e.g, a biological surface) and/or removing biofilm provided herein, or combination provided herein. In certain aspects, the device can be a wound dressing, a topical patch, a syringe, an inhaler, a dosage cup, a dropper, a pump, a spray bottle, an aerosol container or an applicator for administering the composition (e.g., pharmaceutical composition) or combination. In some aspects, the device is a pump for irrigation of a wound or sore with the composition (e.g., pharmaceutical composition) or combination. In aspects, the device is a spray bottle or aerosol container for coating a wound or sore with the composition (e.g., pharmaceutical composition) or combination.

In certain aspects, provided herein are kits that include a polypeptide or a composition provided herein, such as pharmaceutical composition, or composition for treating a surface (e.g, a biological surface) and/or removing biofilm provided herein, or combination provided herein, and a device for administration of the composition. In some aspects, the composition or combination is contained in the device for administration. In certain aspects, the composition or combination is present as a separate component that is distinct from the device.

In certain aspects, the device included in the kits provided herein can be selected from among a dressing, a topical patch, a pump, a spray bottle, an aerosol container, a syringe, an inhaler, a dosage cup, a dropper, or an applicator.

Also provided herein are polynucleotides encoding a polypeptide that includes the sequence of amino acids set forth in SEQ ID NO:1 and/or includes a consecutive sequence of amino acids that is 80% or more, or 90% or more identical to a corresponding consecutive sequence of amino acids set forth in SEQ ID NO:1, wherein the polynucleotide is: (i) a polynucleotide of SEQ ID NO:2, (ii) a polynucleotide that encodes a consecutive sequence of amino acids that is 80% or more, or 90% or more identical to a consecutive sequence of amino acids set forth in SEQ ID NO:1, (iii) a polynucleotide of SEQ ID NO:3, or (iv) a polynucleotide that encodes a consecutive sequence of amino acids that is 80% or more, or 90% or more identical to a consecutive sequence of amino acids set forth in SEQ ID NO:1. Also provided herein are polynucleotides encoding a polypeptide that includes the sequence of amino acids set forth in SEQ ID NO:1 and/or includes a consecutive sequence of amino acids that is 80% or more, or 90% or more identical to a corresponding consecutive sequence of amino acids set forth in SEQ ID NO: 1, wherein the polynucleotide is: (i) a polynucleotide that is 80% or more identical, or 90% or more identical to the sequence of nucleotides set forth in SEQ ID NO:2 or (ii) a polynucleotide that is 80% or more identical, or 90% or more identical to the sequence of nucleotides set forth in SEQ ID NO:3. In certain aspects, the polypeptide encoded by the polynucleotides provided herein exhibits biofilm removal activity and/or enzyme activity.

Also provided herein are vectors containing the polynucleotides provided herein. In certain aspects, a vector provided herein is an expression vector. In aspects, the vector includes the sequence of nucleotides set forth in SEQ ID NO:3.

Provided herein are methods of removing biofilm and/or treating (e.g., disinfecting) a surface that includes contacting the surface or biofilm with, or applying to the surface or biofilm, one or more polypeptides provided herein or a composition provided herein, such as pharmaceutical composition, or composition for treating a surface and/or removing biofilm provided herein, or combination provided herein. In certain aspects, of the methods of removing biofilm and/or treating (e.g., disinfecting) a surface, the one or more polypeptides, or the composition or combination, includes one or more polypeptides exhibiting biofilm removal activity and/or enzyme activity and selected from among (i) a polypeptide of SEQ ID NO:1, (ii) a portion of the polypeptide of SEQ ID NO:1, wherein the portion exhibits biofilm removal activity and/or enzyme activity, and/or (iii) a polypeptide that includes a consecutive sequence of amino acids that is 80% or more, or 90% or more identical to a consecutive sequence of amino acids set forth in SEQ ID NO:1 and exhibits biofilm removal activity and/or enzyme activity. In certain aspects, the surface can be a hard surface, or can be a soft surface, or can be a porous surface. In aspects the surface is a biological surface (or surface of a living substance) or a surface of an inanimate object. In aspects, the surface is a porous surface and the porous surface is selected from among skin, keratin and internal organs. In some aspects, treating (e.g., disinfection), or removing biofilm can include one or more of biofilm removal activity, microbiostatic activity (e.g., bacteriostatic activity) and 15microbicidal activity (e.g., bactericidal activity). In certain aspects, the treating (e.g., disinfection) consists of biofilm removal activity. In aspects, treating (e.g., disinfection) or removing biofilm includes biofilm removal activity and one or more of microbiostatic activity (e.g., bacteriostatic activity) and microbicidal activity (e.g., bactericidal activity).

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIG. 1 shows efficacy of biofilm removal by Polypeptide Q2 as measured in terms of percent removal of biofilm (y-axis).

### DETAILED DESCRIPTION

Polypeptides

Provided herein are polypeptides that include the polypeptide of SEQ ID NO:1 (referred to herein as “Polypeptide Q2”), and polypeptides that include a consecutive sequence of amino acids that is 80% or more, or 90% or more identical to a corresponding consecutive sequence of amino acids of Polypeptide Q2. For example, a consecutive sequence of amino acids of the polypeptides in the compositions provided herein can have at least 80%, 81%, 82%, 83%, 84%, 85%, 86%, 87%, 88%, 89%, 90%, 91%, 92%, 93%, 94%, 95%, 96%, 97%, 98% or 99% or more amino acid sequence identity to a corresponding consecutive sequence of amino acids of Polypeptide Q2 or at least 90%, 91%, 92%, 93%, 94%, 95%, 96%, 97%, 98% or 99% or more amino acid sequence identity to a corresponding consecutive sequence of amino acids of Polypeptide Q2. In some aspects, a polypeptide is an isolated and/or purified polypeptide, a recombinant polypeptide or a synthetically produced polypeptide.

The polypeptides can be isolated or otherwise obtained by methods known to those of skill in the art and/or provided herein. For example, the polypeptides can be generated recombinantly or produced using synthetic processes, such as chemical peptide synthesis methods. In addition, for the polypeptides that have a consecutive sequence of amino acids that bears 80% or more or 90% or more sequence identity to a corresponding consecutive sequence of amino acids of Polypeptide Q2, the modified polypeptides and encoding nucleic acid molecules can include conservative or radical (non-conservative) amino acid substitutions, insertions or deletions. In certain aspects, the modified polypeptides can be produced by standard recombinant DNA techniques known to one of skill in the art and described elsewhere herein. Such modified polypeptides for use in the methods and compositions provided herein also can include modifications of Polypeptide Q2 and the other polypeptides provided herein in a manner that improves stability or half-life, e.g., by chemical modification or a post-translational modification, glycosylation, carboxylation, hydroxylation, sulfation, phosphorylation, albumination, farnesylation, multimerization, conjugation to another protein or polypeptide, such as an antibody or antigen-binding fragment thereof, or conjugation to a polymer such as dextran, a polyethylene glycol (pegylation(PEG)) or sialyl moiety, or other such polymers, such as natural or sugar polymers, and other protein modifications known to those of skill in the art. In addition, one or more amino acids (e.g., a consecutive seqeunce of more than one amino acid) can be included in the polypeptides provided herein to facilitate handling (e.g., isolation, purification) and/or analysis of the polypeptide. Such amino acid(s) additions include tags or other moieties, for example, to aid in detection or affinity purification of the polypeptide. Examples include, but are not limited to, amino acid sequences that can serve as an epitope tag or other detectable marker. Particular non-limiting examples of such sequences include a His tag e.g., 6×His, or a Flag Tag, which are described further in the Examples herein.

The polypeptides provided herein can be used in methods, e.g., for the treatment of a disease or condition susceptible to, associated with and/or accompanied by infection, a tissue, such as animal (e.g., human) tissue, and/or tissue (e.g., surface tissue) injury or damage, including, for example, wounds, sores or burns in or on a subject, or for removing biofilm, or treating (e.g., disinfecting) a surface, e.g., in industrial, dental or health care settings. The polypeptides provided herein also can be used in a composition provided herein, such as pharmaceutical composition, or composition for Treating a surface (e.g, a biological surface) and/or removing biofilm provided herein, or combination provided herein, for use in these methods.

Compositions and Combinations

Certain aspects of the compositions and combinations provided herein are now described. In some aspects, provided are compositions and combinations for treating a disease or condition susceptible to, characterized, associated with and/or accompanied by infection, e.g. microbial infection. The microbial infections can be associated with the mucosa, e.g., peptidic ulcers in the stomach lining or infections associated with lung mucosa in cystic fibrosis or tuberculosis, or the infections can be associated with wounds or sores resulting from a disease or condition. Also provided are compositions and combinations for treating, e.g., decontaminating /disinfecting surfaces, for example, in industrial, dental or health care settings. In certain aspects, the compositions and combinations provided herein can kill, inhibit the growth of, or otherwise eliminate microbes that are associated with infections, wounds or sores, or microbes that are associated with certain surfaces such biological surfaces (e.g., surface of a living tissue) and surfaces of inanimate objects, such as in industrial, dental or health care settings. In some aspects, the compositions and combinations provided herein can render the microbes susceptible to traditional approaches, e.g., the use of antibiotics, antifungal agents, antiviral agents, antiseptics and/or oxidizing biocides that can kill the microbes and/or inhibit their growth and/or eliminate them, e.g., from a surface. For example, in subjects having diabetic sores associated with diabetes, e.g., diabetes mellitus, treatment with the compositions provided herein can increase the potency of other treatments, e.g., antibiotics, by providing access to the underlying tissues in the sore. In some aspects, the compositions and combinations provided herein can reduce the impact of viruses, such as the viruses responsible for COVID-19, shingles, warts, and cold sores.

The compositions and combinations provided herein can include one or more polypeptides selected from among the polypeptide of SEQ ID NO:1 (referred to herein as “Polypeptide Q2”), and polypeptides that include a consecutive sequence of amino acids that is 80% or more, or 90% or more identical to a corresponding consecutive sequence of amino acids of Polypeptide Q2. For example, a consecutive sequence of amino acids of the polypeptides in the compositions provided herein can have at least 80%, 81%, 82%, 83%, 84%, 85%, 86%, 87%, 88%, 89%, 90%, 9192%, 93%, 94%, 95%, 96%, 97%, 98% or 99% or more amino acid sequence identity to a corresponding consecutive sequence of amino acids of Polypeptide Q2 or at least 90%, 91%, 92%, 93%, 94%, 95%, 96%, 97%, 98% or 99% or more amino acid sequence identity to a corresponding consecutive sequence of amino acids of Polypeptide Q2. In certain aspects, the polypeptide is an isolated and/or purified, recombinant or synthetically 15produced polypeptide. A recombinant polypeptide is one that is produced using genetic recombination/engineering technologies and can involve heterologous expression of a polynucleotide encoding the polypeptide in a host cell system, e.g., heterologous host cell. A synthetically produced polypeptide is one that is generated using synthetic chemistry methods. The compositions and combinations provided herein can be pharmaceutical compositions or combinations, e.g., for the treatment of diseases or conditions that are accompanied by infections, wounds or sores in or on a subject, or they can be compositions or combinations for removing biofilm or treating (e.g., disinfecting) surfaces, e.g., in industrial, dental or health care settings.

The compositions and combinations provided herein and/or one or more polypeptide components of the compositions and combinations provided herein can, in certain aspects, have microbicidal activity and/or microbiostatic activity. In certain aspects, the microbes are bacteria, fungi or viruses and the compositions and combinations provided herein and/or one or more polypeptide components of the compositions provided herein can have bactericidal activity and/or bacteriostatic activity, fungicidal and/or fungistatic activity, viricidal activity, and/or can reduce the impact of viral infections.

In aspects, the compositions and combinations provided herein and/or one or more polypeptide components of the compositions and combinations provided herein can have biofilm removal activity and/or enzyme activity. In some embodiments, the enzyme activity includes a hydrolase activity, such as, for example, a hydrolase activity that acts on carboxylic ester bonds, e.g., an esterase, such as, for example, a serine esterase. In some aspects, the enzyme activity includes a cutinase activity. As described elsewhere herein, biofilms contain an attached community of microorganisms embedded in an exopolymer matrix that often persists despite attempts with traditional approaches designed to kill free-floating microorganisms because biofilms often are resistant to such treatments, e.g., with antibiotics, antiseptics, and oxidizing biocides. In addition, biofilms often confer immune resistance or create barriers to treatment of the underlying disease or condition. Thus, in certain aspects, the compositions and combinations provided herein are for removing biofilm barriers and increasing the potency of other therapeutically active agents or surface treatment agents for treating a surface or treating an underlying disease or condition, or an infection, wound or sore associated with an underlying disease or condition. The compositions and combinations having biofilm removal activity, as provided herein, also can, in certain aspects, include additional agents for biofilm removal, such as those described, for example, in WO/2006031554, WO/2001098214, WO/1998026807, WO/2004041988, WO/1999014312 and WO/2001053010.

In certain aspects, the compositions and combinations provided herein and/or one or more polypeptide components of the compositions and combinations provided herein can have biofilm removal activity and one or both of microbicidal activity and microbiostatic activity, for example, bactericidal activity. In aspects, any of the compositions, e.g., pharmaceutical compositions, such as pharmaceutical compositions, or compositions for treating a surface and/or removing biofilm provided herein, or combinations provided herein, can include one or more additional surface treatment agents, and/or therapeutically active agents for treating a surface or an underlying disease or condition, or for treating an infection, wound or sore associated with the underlying disease or condition.

The polypeptides contained in the compositions provided herein can be isolated from a microbial organism, such as, for example, a bacterium or fungus. The bacterium or fungus can be a thermophilic microorganism. For example, Polypeptide Q2 can be isolated from the thermophilic fungus, TM-417. Thus, in certain aspects, the compositions provided herein and/or one or more polypeptide components of the compositions provided herein are thermally stable.

Methods for production and purification of proteins such as Polypeptide Q2 for use in the compositions, including pharmaceutical compositions, and methods provided herein are known in the art (see, e.g., Tan et al., Biomed. Res. Int, Article ID 574398 (2009)). In addition, for polypeptide components of the compositions provided herein that have a consecutive sequence of amino acids that bears 90% or more sequence identity to a corresponding consecutive sequence of amino acids of Polypeptide Q2, the modified polypeptides and encoding nucleic acid molecules can be produced by standard recombinant DNA techniques known to one of skill in the art. Any method known in the art to effect mutation of any one or more amino acids in a target protein, e.g., Polypeptide Q2, can be employed. Methods can include standard site-directed or random mutagenesis of encoding nucleic acid molecules, or solid phase polypeptide synthesis methods. For example, nucleic acid molecules encoding a Polypeptide Q2 polypeptide can be subjected to mutagenesis, such as random mutagenesis of the encoding nucleic acid, error-prone PCR, site-directed mutagenesis, overlap PCR, gene shuffling, or other recombinant methods. The nucleic acids encoding the polypeptides can then be introduced into a host cell to be expressed heterologously. Hence, also provided herein are nucleic acid molecules encoding any of the polypeptides of the compositions provided herein. In some aspects, the polypeptides of the compositions provided herein can be produced synthetically, such as by using solid phase or solution phase peptide synthesis.

Polypeptides such as Polypeptide Q2 also can be obtained by methods well known in the art for recombinant protein expression and purification. An example of a method for recombinant expression and purification of Polypeptide Q2 is provided in Example 1. Any method known to those of skill in the art for identification of nucleic acids that encode desired genes can be used. Any method available in the art can be used to obtain a full length (i.e., encompassing the entire coding region) cDNA or genomic DNA clone encoding a polypeptide provided herein, such as from a cell or tissue source. Modified Polypeptide Q2 polypeptides, such as those bearing 80% or more, or 90% or more consecutive amino acid sequence identity with a corresponding consecutive amino acid sequence in Polypeptide Q2, can be engineered from a wildtype polypeptide, such as by site-directed mutagenesis.

The polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, such as Polypeptide Q2, including for example, polymerase chain reaction (PCR) methods. A nucleic acid containing material can be used as a starting material from which a desired polypeptide-encoding nucleic acid molecule can be isolated. For example, DNA and mRNA preparations, or crude microbial extracts, such as a TM-417 fungal extract, can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide. A polynucleotide encoding Polypeptide Q2 obtained by amplification of nucleic acids from TM-417 fungal cells is provided herein as SEQ ID NO:2.

A polynucleotide encoding a polypeptide provided herein, e.g., Polypeptide Q2, can also be synthetically produced using methods known in the art, such as, for example, synthetic chemistry based polynucleotide synthesis methods. Additionally, the polynucleotides encoding a polypeptide provided herein can be modified for any reason, including, for example, to facilitate or enhance expression in a recombinant host. For example, a polynucleotide sequence encoding a polypeptide provided herein can be modified to optimize the codons encoding the amino sequence of the desired polypeptide without altering the amino sequence. The nucleotide sequence of a polynucleotide encoding Polypeptide Q2 which is codon optimized for insertion in plasmid DNA is provided herein in SEQ ID NO:3.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. Additional nucleotide residues sequences such as sequences of bases specifying protein binding regions also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene.

In addition, tags or other moieties can be added, for example, to aid in detection or affinity purification of the polypeptide. For example, additional nucleotide residues sequences such as sequences of bases specifying an epitope tag or other detectable marker also can be linked to enzyme-encoding nucleic acid molecules. Non-limiting examples of such sequences include nucleic acid sequences encoding a His tag e.g., 6×His, or a Flag Tag.

Also provided herein are polynucleotides encoding a polypeptide that includes the sequence of amino acids set forth in SEQ ID NO:1 and/or includes a consecutive sequence of amino acids that is 80% or more, or 90% or more identical to a corresponding consecutive sequence of amino acids set forth in SEQ ID NO:1, wherein the polynucleotide is: (i) a polynucleotide of SEQ ID NO:2, (ii) a polynucleotide that encodes a consecutive sequence of amino acids that is 80% or more, or 90% or more identical to a consecutive sequence of amino acids set forth in SEQ ID NO:1, (iii) a polynucleotide of SEQ ID NO:3, or (iv) a polynucleotide that encodes a consecutive sequence of amino acids that is 80% or more, or 90% or more identical to a consecutive sequence of amino acids set forth in SEQ ID NO:1. Also provided herein are polynucleotides encoding a polypeptide that includes the sequence of amino acids set forth in SEQ ID NO:1 and/or includes a consecutive sequence of amino acids that is 80% or more, or 90% or more identical to a corresponding consecutive sequence of amino acids set forth in SEQ ID NO: 1, wherein the polynucleotide is: (i) a polynucleotide that is 80% or more identical, or 90% or more identical to the sequence of nucleotides set forth in SEQ ID NO:2 or (ii) a polynucleotide that is 80% or more identical, or 90% or more identical to the sequence of nucleotides set forth in SEQ ID NO:3. In certain aspects, the polypeptide encoded by the polynucleotides provided herein exhibits biofilm removal activity and/or enzyme activity. In aspects, the biofilm removal activity and/or enzyme activity is thermally stable. In some aspects, a polynucleotide provided herein encodes a consecutive sequence of amino acids that has at least 80%, 81%, 82%, 83%, 84%, 85%, 86%, 87%, 88%, 89%, 90%, 91%, 92%, 93%, 94%, 95%, 96%, 97%, 98% or 99% or more amino acid sequence identity to a corresponding consecutive sequence of amino acids of SEQ ID NO: 1, or at least 90%, 91%, 92%, 93%, 94%, 95%, 96%, 97%, 98% or 99% or more amino acid sequence identity to a corresponding consecutive sequence of amino acids of SEQ ID NO:1. In some aspects, such a polynucleotide is at least 80%, 81%, 82%, 83%, 84%, 85%, 86%, 87%, 88%, 89%, 90%, 91%, 92%, 93%, 94%, 95%, 96%, 97%, 98% or 99% identical to the sequence of nucleotides set forth in SEQ ID NO:2 or SEQ ID NO:3, or at least 90%, 91%, 92%, 93%, 94%, 95%, 96%, 97%, 98% or 99% identical to the sequence of nucleotides set forth in SEQ ID NO:2 or SEQ ID NO:3.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the HZ24 expression vector. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be 25produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

For recombinant expression of a protein such as Polypeptide Q2, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions. Also provided are vectors that contain a nucleic acid encoding Polypeptide Q2 and variants that bear 80% or more, or 90% or more sequence identity thereto, as provided in the compositions described herein. Cells containing the vectors also are provided. The cells include eukaryotic and prokaryotic cells, and the vectors are any suitable for use therein.

Prokaryotic and eukaryotic cells containing the vectors are provided. Such cells include bacterial cells, yeast cells, fungal cells, Archea, plant cells, insect cells and animal cells. The cells are used to produce a protein thereof by growing the above-described cells under conditions whereby the encoded polypeptide is expressed by the cell, and recovering the expressed polypeptide.

Provided are vectors that contain a sequence of nucleotides that encodes Polypeptide Q2, or a polypeptide having a consecutive sequence of amino acids that is 80% or more, or 90% or more identical to a corresponding consecutive sequence of amino acids of Polypeptide Q2. The vectors can be selected for expression of the polypeptide in the cell or such that the polypeptide is expressed as a secreted polypeptide.

A variety of host-vector systems can be used to express the coding sequences of the polypeptides of the compositions provided herein. These include, but are not limited to, mammalian cell systems; insect cell systems; microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the polypeptides can be controlled by any promoter/enhancer known in the art. Promoters that can be used include, but are not limited to, the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25(1983)); see also “Useful Proteins from Recombinant Bacteria”: in Scientific American 242:79-94 (1980)); plant expression vectors containing the nopaline synthetase promoter (Herrera-Estrella et al., Nature 303:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter and the alkaline phosphatase promoter.

The compositions and combinations provided herein can be formulated for direct administration, or application, or can require dilution. They can be formulated for multiple or single dosage (or application) administration. Non-limiting examples of compositions include concentrations of polypeptide(s) e.g., Polypeptide Q2 and/or and variants that bear 80% or more, or 90% or more sequence identity thereto, of between about 0.1 μg/mL to 400 μg/mL, 1 μg/mL to 350 μg/mL, 5 μg/mL to 350 μg/mL, 5 μg/mL to 250 μg/mL, 5 μg/mL to 100 μg/mL, 10μg/mL to 50 μg/mL, 100 μg/mL to 350 μg/mL, 150 μg/mL to 300 μg/mL or 5 μg/mL to 40 μg/mL. Non-limiting examples of compositions also include concentrations of polypeptide(s) provided herein, e.g., Polypeptide Q2 and/or and variants that bear 80% or more, or 90% or more sequence identity thereto, of about 0.1% to about 10%, about 0.1% to about 9%, about 0.1% to about 8%, about 0.1% to about 7%, about 0.1% to about 6%, about 0.1% to about 5%, about 0.1% to 30about 4%, about 0.1% to about 3%, about 0.1% to about 2%, or about 0.1% to about 1% weight/volume of the one or more polypeptides. Further non-limiting examples of compositions also include concentrations of polypeptide(s) provided herein, e.g., Polypeptide Q2 and/or and variants that bear 80% or more, or 90% or more sequence identity thereto, of about 10% or less, 9% or less, 8% or less, 7% or less, 6% or less, 5% or less, 4% or less, 3% or less, 2% or less, 1% or less, 0.5% or less, or 0.25% or less weight/volume of the one or more polypeptides.

In certain aspects, the compositions and combinations provided herein have biofilm removal activity and the concentrations of polypeptide(s) in the compositions or combinations provided herein are in an amount that reduces the amount of biofilm in a subject or on a surface by about 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more, up to 100%. In vitro assays using biofilm-specific staining to measure the amount of biofilm are known to those of skill in the art. An example of an in vitro assay for measuring biofilm removal activity is demonstrated in Example 2. For example, biofilm removal can be measured using a crystal violet assay (see, e.g., U.S. Pat. No. 8,597,927). Samples are immersed in a solution of Crystal Violet (0.31% w/v) for ten minutes prior to rinsing three times in phosphate buffered saline (PBS) to remove unbound stain. Bound stain retained by a 15biofilm is extracted with 95% ethanol and the absorbance of the Crystal Violet/ethanol solution is read at 540 nm. Percent removal of a biofilm is calculated as [(1-Fraction remaining biofilm)×100]. Fraction remaining biofilm is calculated by subtracting the absorbance of the medium +enzyme solutions from the absorbance of the solutions extracted from the enzyme treated biofilms, divided by the difference in absorbance from that of untreated control biofilms minus the absorbance of the growth medium only. Other biofilm-specific stains that can be used include, but are not limited to, Film Tracer (Thermo Fisher Scientific, Inc.), Safranin, Congo Red, Acridine Orange and histochemical staining.

Biofilm removal also can be assessed by suspending cells from a treated biofilm in a buffer such as phosphate buffered saline (PBS) by plating cells on a suitable nutrient containing growth medium and counting the number of cells that grow on the medium. Reduction in viable cell count can be determined by comparison with the number of cells that grow from a suspension prepared from a control untreated biofilm.

An example of an in vitro assay for measuring biofilm removal activity is demonstrated in Example 2. In vivo models for measuring biofilms also are known in the art (see, e.g., Seth et al., J. Surg. Res., 178:330-338 (2012), and documents cited therein).

In certain aspects, the compositions provided herein have bactericidal and/or bacteriostatic activity and the concentrations of polypeptide(s) in the compositions provided herein are in an amount that kills or inhibits the growth of, or reduces the number of, bacteria in a subject or on a surface by about 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more, up to 100%.

In some aspects, the compositions can take the form of solutions, suspensions, emulsions, tablets, pills, capsules, powders, and sustained release formulations. Oral formulations, e.g., for pharmaceutical compositions, can include standard carriers such as pharmaceutical grades of mannitol, lactose, starch, magnesium stearate, sodium saccharine, cellulose, magnesium carbonate, and other such agents. Topical formulations also are contemplated.

In certain aspects, the compositions or combinations are pharmaceutical compositions or combinations that are co-formulated with one or more additional therapeutically active agents including, but not limited to, a chemotherapeutic agent, an analgesic agent, an antibiotic, an anti-inflammatory agent, an antimicrobial agent, an amoebicidal agent, a trichomonacidal agent, an anti-Parkinson agent, an anti-malarial agent, an anticonvulsant agent, an anti-depressant agent, and antiarthritics agent, an anti-fungal agent, an antiviral agent, an antihypertensive agent, an antipyretic agent, an anti-parasite agent, an antihistamine agent, an alpha-adrenergic agonist agent, an alpha blocker agent, an anesthetic agent, a bronchial dilator agent, a biocide agent, a bactericide agent, a bacteriostat agent, a beta adrenergic blocker agent, a calcium channel blocker agent, a cardiovascular drug agent, a contraceptive agent, a decongestant agent, a diuretic agent, a depressant agent, a diagnostic agent, a electrolyte agent, a hypnotic agent, a hormone agent, a hyperglycemic agent, a muscle relaxant agent, a muscle contracting agent, an ophthalmic agent, a parasympathomimetic agent, a psychic energizer agent, a sedative agent, a sympathomimetic agent, a tranquilizer agent, an urinary agent, a vaginal agent, a viricide agent, a vitamin agent, a non-steroidal anti-inflammatory agent, an angiotensin converting enzyme inhibitor agent, a polypeptide, a protein, a nucleic acid, a drug, an organic molecule or a sleep inducer. In some aspects, the aforementioned additional therapeutically active agents are independently formulated and are administered sequentially, serially, simultaneously, concurrently or intermittently with the pharmaceutical compositions, or in the combinations, provided herein.

Any of the compositions and combinations provided herein can be administered using a device. In certain aspects, the device can be a wound dressing, a topical patch, a syringe, an inhaler, a dosage cup, a dropper, a pump, a spray bottle, an aerosol container or an applicator for administering the composition, e.g., pharmaceutical composition or combination, e.g., pharmaceutical combination. In some aspects, the device is a pump for irrigation of a wound or sore with the composition, e.g., pharmaceutical composition, or combination. In aspects, the device is a spray bottle or aerosol container for coating a wound or sore with the composition, e.g., pharmaceutical composition, or combination, e.g., pharmaceutical combination. Also provided herein are kits that include compositions or combinations provided herein and a device for administration of the compositions or combinations.

Methods

Certain aspects of the methods provided herein are now described. In certain aspects, provided herein are methods of treating a disease or condition in a subject that includes administering, to a subject in need thereof, a composition, e.g., pharmaceutical composition or a composition for removing biofilm or treating a surface, provided herein or a combination, provided herein. In some aspects, the methods include administering a therapeutically effective amount of a composition, e.g., pharmaceutical composition, or combination provided herein. In some aspects, the disease or condition is one that is susceptible to, associated with and/or accompanied by an infection, tissue, such as animal (e.g., human) tissue, and/or tissue (e.g., surface tissue) injury or damage, including, for example, wounds, sores or burns in or on a subject. In some aspects, the disease or condition is accompanied by or associated with tissue, including, for example, epithelial tissue, connective tissue, or tissue membranes (e.g., mucous membranes, cutaneous membranes and serous membranes). In certain aspects, the disease or condition can be associated with mucosa, a wound, sore, ulcer or infection. Non-limiting examples of diseases or conditions that can be treated using the pharmaceutical compositions provided herein are described below:

Cystic Fibrosis

Cystic fibrosis, a genetically inherited disease, is caused by the mutation of a gene that produces an electrolyte transfer protein. The consequence of the mutation affects a multitude of organ systems. However, the tissues that are most directly affected are those that secrete mucus or have mucus membranes. Adverse consequences occur with tissues that are associated with the respiratory system i.e., lungs and airway passage tissues. In response to the genetic defect, the host produces secretions to counteract the ionic imbalance. These secretions in response to the disease allow opportunistic infections to develop, which causes additional fluid and mucus to infiltrate the respiratory system from the host. The infectious bacteria and the associated bacterial biofilm add additional mucus and fluid at the site. In addition, the principal bacterial pathogen associated with the opportunistic infection is Pseudomonas aeruginosa, which often mutates to a mucoid form that is a prolific producer of alginate biofilm and further exacerbates the disease condition. The result is an accumulation of copious amount of mucus, fluid and biofilm material which affects not only the respiratory system, but the entire host.

The current treatment of cystic fibrosis involves a dual approach to: 1) promote and facilitate the removal of mucus and secretions from the respiratory tract and; 2) control the infection that is associated with the disease. The infection, with the bacteria's production of a biofilm, obstructs the host's defenses, shields the bacteria from the killing action of antibiotics and increases the viscosity of the mucus, making it increasingly difficult for the patient to expectorate the interfering mucus. Thus, provided herein are compositions, e.g., pharmaceutical compositions, and combinations, e.g., pharmaceutical combinations, that control the infection associated with cystic fibrosis and increase responsiveness, e.g., to the action of antibiotics.

Infection

Wound infection can lead to the interruption of several processes along the wound healing pathway. Bacteria produce inflammatory mediators that inhibit the inflammatory phase as well as epithelialization phase of wound healing. Bacteria in an infected wound cause cell death, which will lead to an increase in local inflammation response and prolonged acute inflammatory phase. The presence of necrotic tissue prevents the ingrowth of new tissue. In addition, necrotic tissue also serves as a culture for bacterial proliferation, therefore, leading to a vicious pathologic cycle. Thus, provided herein are compositions, e.g., pharmaceutical compositions, and combinations, e.g., pharmaceutical combinations, that treat wound infection or increase the susceptibility of wound infection to traditional treatment approaches, e.g., the action of antibiotics.

Also provided herein are compositions, e.g., pharmaceutical compositions, and combinations, e.g., pharmaceutical combinations, for the treatment of infections, such as tuberculosis or COVID-19.

Izschemic Conditions

Ischemia is a condition that results in chronic non-healing wounds and can be seen in arterial insufficiency, venous hypertension, and pressure injuries.

Ischemic Condition Due to Arterial Insufficiency

In subjects with arterial insufficiency, arterial blood flow to the tissue is diminished, leading to a decrease in the delivery of oxygen and nutrients to the wound, and impairment in the removal of metabolic waste products from the wound. Limb threatening ischemia develops when blood flow to the extremity is insufficient to meet the metabolic demands of the tissue, manifesting as rest pain or non-healing wounds. Due to the chronic deficiency of oxygen and nutrients, the skin of the affected limb is not able to maintain normal tissue architectures. Clinically, this is manifested as shiny skin surface with paucity of hair. The metabolic demands to maintain intact skin is higher than the metabolic demands to heal a wound. A simple cut or abrasion on the skin caused by poor fitting shoes can alter the balance of metabolic demand, leading to a chronic wound. The most common cause of arterial insufficiency ulcers is obstruction of large and medium-size arteries caused by atherosclerosis. Other conditions that affect small arteries such as vasculitis, thromboangiitis obliterans, and scleroderma can also cause ischemic ulcers. Provided herein are compositions, e.g., pharmaceutical compositions, and combinations, e.g., pharmaceutical combinations, that treat arterial insufficiency ulcers or increase the susceptibility of these ulcers to traditional treatment approaches.

Ischemic condition due to venous hypertension

In subjects with chronic deep vein thrombosis, arteriovenous fistula or venous insufficiency, hydrostatic pressure is built up in the venous system leading to venous hypertension. This results in the loss of pressure gradient between the arterioles and the venules, leading to slowing of the movement of blood within the capillaries. This sluggish movement of blood results in sequestration of erythrocytes and leukocytes within the capillaries, and the elevation of hydrostatic pressure within the capillaries results in capillary leak. The fibrinogen leaked from the capillaries of the dermis form a fibrin cuff. This fibrin cuff, in combination with tissue edema, result in decreased oxygen permeability, leading to tissue hypoxia and impaired wound healing. The leukocytes that are trapped in the capillaries adhere to the endothelium and release inflammatory mediators and reactive oxygen metabolites. These, in turn, cause endothelial damage, obliteration of the capillaries, and subsequent tissue ischemia. Capillaries of subjects with venous stasis are also occluded by microthrombi that, in turn, reduce oxygen and nutrition to the tissue, predisposing to ulcer formation. Provided herein are compositions, e.g., pharmaceutical compositions, and combinations, e.g., pharmaceutical combinations, that treat venous hypertension ulcers or increase the susceptibility of these ulcers to traditional treatment approaches.

Ischemic Condition Due to Pressure Injries

The development of pressure injuries is caused by a combination of pressure and shear force. Pressure is defined as force per unit area. Therefore, smaller areas such those over bony prominences sustain higher pressure and are at higher risk for development of pressure injuries. When the pressure applied to the skin is in excess of 1rteriolar pressure, oxygen and nutrient delivery to the tissue is shut off, resulting in tissue hypoxia and accumulation of waste products and free radicals. In animal models, pressure in excess of 70 mm of Hg for two hours result in irreversible tissue damage. Although critical duration of ischemia can vary among individuals and clinical situations, as a rule of thumb, pressure injuries can develop within 1 to 4 hours of sustained 2ressure load. Beside the externally applied pressure, the individual tolerance of tissue to ischemia also plays an important role. Evidence suggests that subjects with peripheral arterial occlusive disease are at higher risk for developing pressure ulcers. Critically ill patients with higher disease severity scores such as Acute Physiology and Chronic Health Evaluation (APACHE) score, are more likely to develop pressure ulcers due to global hypoperfusion of tissue, predisposing them to ischemic pressure injuries. Extensive tissue damage can sometimes occur with little evidence of superficial injury. Shear force occurs when there is lateral displacement of the tissue, usually by gravity or by subjects being dragged across an external surface. In these situations, deeper tissues such as bone, muscles, and subcutaneous fat are shifted laterally while the dermis remain fixed through contact with external surfaces. The result of this lateral displacement is injury to the tissue, blood vessels, and lymphatics. Excessive moisture in the form of perspiration, urine, and feces can lead to maceration of the superficial tissue and changes in the cutaneous chemical environment. Although excessive moisture by itself does not directly cause pressure injuries, it can lead to impaired wound healing and promote the generation of chronic wounds. Provided herein are compositions, e.g., pharmaceutical compositions, and combinations, e.g., pharmaceutical combinations, that treat wounds occurring as a result of pressure injuries or increase their susceptibility to traditional treatment approaches.

Metabolic Conditions

Certain metabolic conditions that contribute to the development of non-healing wounds.

Diabetes Mellitus

There are several factors related to diabetes that can contribute to the pathogenesis of diabetic foot ulcers. In diabetic neuropathy, damage to the sensory, motor, and autonomic nerve fibers affects peripheral sensation, motor innervation of small muscles in the foot, and fine vasomotor control of the pedal circulation. In sensory neuropathy, the loss of protective sensation leads to lack of awareness of sustained pressure on the tissue or injury to the tissue. Motor neuropathy usually affects the innervation of the small intrinsic muscles of the feet, resulting in the unopposed action of the larger muscles in the anterior tibial compartment. This leads to subluxation of the proximal metatarsal-phalangeal joints, giving the feet the appearance of claw toes. Consequently, the pressure is redistributed abnormally to the metatarsal heads, where reactive thickening of the skin (callus) forms. Ischemic necrosis of the tissue under the callused skin eventually leads to breakdown of the skin, resulting in a neuropathic ulcer with the punched-out appearance commonly under the metatarsal heads. Autonomic neuropathy is characterized by a lack of autonomic tone in the arteriolar and capillary circulation, causing shunting of blood from the arterioles directly to the veins, thus bypassing the tissue that needs the nutrients. Although the feet may have bounding pulses and distended veins, the tissue can lack the perfusion needed for healing and to fight infection. Additionally, the loss of autonomic innervation to the sweat glands of the feet results in dry, scaly skin that can crack easily, allowing bacteria to penetrate. The Charcot foot is a later complication of diabetes and is characterized by collapse of the arch of the midfoot. Osteopenia due to arteriolar-venous shunting of autonomic neuropathy combined with small intrinsic muscle wasting, lead to loss of structural integrity of the bone. Stress and minor trauma can induce fracture of a weakened bone, putting more stress on the adjacent weakened bones, and the cycle repeats, ultimately leading to gross deformity. The resulting abnormal bony prominences that form greatly increase the risk of ulceration. Provided herein are compositions, e.g., pharmaceutical compositions, and combinations, e.g., pharmaceutical combinations, that treat diabetic sores or ulcers or increase the susceptibility of these sores or ulcers to traditional treatment approaches.

Malnutrition

Fatty acids are essential for the inflammatory phase of wound healing as they provide the arachidonic acid substrate for eicosanoid synthesis. Proteins are required for the proliferation of fibroblasts and the synthesis and deposition of collagen. Proteins are also required for important immunologic functions such as phagocytic activity of macrophages, T-cell function, and compliment and antibody production. Malnutrition can impair wound healing by prolonging the inflammatory phase and by reducing the proliferation of fibroblasts and deposition of collagen. Malnutrition is associated with increased risk of wound infection, which has deleterious effects on wound healing. Provided herein are compositions, e.g., pharmaceutical compositions, and combinations, e.g., pharmaceutical combinations, that treat wounds associated with malnutrition or increase the susceptibility of these wounds to traditional treatment approaches.

Immunosuppression

Systemic immunosuppression can contribute to the development of non-healing wounds. Corticosteroids have been shown to interfere with all major steps of the wound healing process in both animals and human studies. In the inflammatory phase, corticosteroid decreases the expression of cytokines that are responsible for the recruitment of inflammatory cells, as well as the expression of adhesion molecules responsible for 25adhesion and migration of granulocytes. In the proliferative phase, corticosteroids reduce the levels of transforming growth factor-β and keratinocyte growth factor, which attenuates fibroblast proliferation and wound epithelization respectively. In the remodeling phase, corticosteroids impair collagen accumulation as well as collagen turnover. Clinically, subjects who are on chronic corticosteroids that undergo surgery have significantly increased infection rate as well as wound dehiscence rate. Immunosuppressive agents can suppress the expression of several inflammatory mediators that are involved in the wound healing process. Immunosuppressive agents inhibit the proliferation of immune cells and may blunt the inflammatory phase of wound healing. Sirolimus, an immunosuppressive agent that inhibits the threonine kinase known as mammalian target of rapamycin (mTOR), is associated with a significantly higher wound complication rate compared to other common immunosuppressive agents.

Chemotherapeutic agents, particularly those that target vascular endothelial growth factor (VEGF) and epidermal growth factor receptor (EGFR), can have detrimental effects on wound healing. Inhibition of VEGF results in the suppression of angiogenesis, which is an important part of wound healing. Epidermal growth factor receptor inhibitors can negatively impact wound healing by inhibiting epithelialization. Provided herein are compositions, e.g., pharmaceutical compositions, and combinations, e.g., pharmaceutical combinations, that treat wounds associated with immunosuppression or increase the susceptibility of these wounds to traditional treatment approaches.

Radiation

Ionized radiation used in the treatment of cancer can cause delay in wound healing. Sometimes, radiation injury results in chronic non-healing wounds. Ionizing radiation causes cellular damage by breaking the double-stranded cellular DNA and releasing free radicals. This effect is most profound in cells undergoing DNA replication and mitosis. The resulting effect is apoptosis and cellular necrosis. Radiation also can cause eccentric myointimal proliferation in the small arteries and arterioles, which may cause luminal thrombosis and obstruction. This results in ischemia to the tissue which can progress to tissue necrosis. Provided herein are compositions, e.g., pharmaceutical compositions, and combinations, e.g., pharmaceutical combinations, that can arrest or inhibit delays caused by radiation treatments in the healing of wounds or increase the susceptibility of treating such delays using traditional approaches.

Decontamination/Disinfection of Surfaces

In certain aspects, provided herein are methods of decontaminating/disinfecting a surface using the compositions provided herein. A “surface,” as used herein, refers to a structure having sufficient mass to allow for attachment of a biofilm. Hard surfaces include, but are not limited to, metal, glass, ceramic, wood, mineral (e.g., rock, stone, marble, granite), aggregate materials such as concrete, plastic, composite material, hard rubber material, and gypsum. A hard material can be finished with enamel and/or paint. Hard surfaces are found, for example, in water treatment and storage equipment and tanks, dairy and food processing equipment and facilities, medical equipment and facilities, e.g., surgical instruments, permanent and temporary implants, and industrial pharmaceutical equipment and plants. Soft surfaces include, but are not limited to, hair and textiles. Porous surfaces can be biological surfaces, including, but not limited to, skin, keratin, mucous membranes, and internal organs. Porous surfaces can also be found in certain ceramics as well as in membranes that are used for filtration. Other surfaces include, but are not limited to, ship hulls and swimming pools.

Exemplary Amino Acid and Nucleotide Sequences

| SEQ ID | |
|---|---|
| NO: | AMINO ACID OR NUCLEOTIDE SEQUENCE |
| 1 | MKGFIAACLALAGLALGAPEVKRQKSDLTPQLLQNFHLFAQWSEAAGCNNQKAAGQPVTCDP |
| | GQCSLFDKHNATVVASFIGDVLDTRGFVGVDHVDKLIVVSFRGTTSVRNWIADLLFFQVSCD |
| | LTPGCLAHAGFYASWEEVKSRVLDAVKKAKAANPSYRVVATGYSLGGAIATLAVAYIRQQGH |
| | AADLYTYGGPRVGNLALAEFITNQPGGEYRITHKDDPVPRLPPLILNYRHTSPEYWIDPGND |
| | RTVSLDEVQYCPGYSNTDCNGGTDGLNLDSHGFYFQSFIGCGPGSTPWRRASDISDKELEDR |
| | VNLFAELDRQLAKSLHEAGEQ |
| 2 | ATGAAGGGTTTCATAGCCGCTTGCCTCGCCCTGGCGGGCCTAGCCCTGGGAGCCCCCGAGGT |
| | CAAGAGGCAGAAGAGCGACCTCACCCCGCAGCTGCTGCAAAACTTCCATCTGTTCGCCCAGT |
| | GGTCCGAGGCGGCTGGGTGCAACAACCAAAAGGCAGCCGGGCAGCCTGTGACCTGCGACCCG |
| | GGCCAGTGCTCTCTGTTCGACAAGCACAACGCCACCGTCGTCGCCTCCTTCATCGGTGACGT |
| | GCTCGACACTCGGGGTTTCGTCGGCGTTGACCATGTGGATAAGCTGATTGTCGTCTCCTTCC |
| | GGGGTACCACCTCGGTGCGGAACTGGATCGCCGACCTCCTCTTCTTCCAAGTGTCCTGCGAC |
| | CTGACTCCCGGCTGCCTCGCCCACGCGGGCTTCTACGCGTCGTGGGAGGAGGTCAAGTCGCG |
| | CGTGCTCGACGCCGTGAAGAAGGCCAAGGCGGCCAACCCGAGCTACCGCGTCGTGGCGACGG |
| | GCTACTCGCTGGGCGGCGCCATCGCCACCTTGGCCGTCGCCTACATCCGCCAGCAAGGGCAC |
| | GCCGCCGACCTGTACACGTACGGCGGCCCGCGCGTCGGCAACCTCGCCCTGGCCGAGTTCAT |
| | CACGAACCAGCCCGGCGGCGAGTACCGCATCACGCACAAGGACGACCCCGTCCCGCGCCTCC |
| | CGCCCTTGATCCTCAACTACCGCCACACGAGCCCCGAGTACTGGATCGACCCGGGCAACGAC |
| | CGCACCGTCTCCCTCGACGAGGTCCAGTACTGCCCGGGCTACTCCAACACGGACTGCAACGG |
| | CGGCACCGACGGCCTCAACCTCGACTCGCACGGCTTCTACTTCCAGAGCTTCATCGGCTGCG |
| | GCCCCGGCAGCACCCCGTGGCGCCGCGCCAGCGATATCAGCGACAAGGAGCTCGAGGACAGG |
| | GTCAACTTGTTTGCCGAGCTCGACAGGCAGCTGGCCAAGAGCCTGCACGAGGCCGGCGAGCA |
| | GTGA |
| 3 | ATGAAAGGATTCATTGCTGCGTGCCTTGCTTTAGCGGGACTTGCGCTTGGAGCGCCCGAGGT |
| | TAAACGTCAAAAGAGTGATCTGACACCCCAGCTTCTTCAAAATTTTCATTTATTTGCCCAGT |
| | GGAGCGAAGCGGCGGGGTGCAATAACCAGAAAGCCGCGGGGCAACCTGTGACATGTGACCCC |
| | GGACAGTGTTCACTGTTCGATAAGCACAACGCAACGGTCGTGGCGAGTTTCATCGGGGACGT |
| | GTTGGACACTCGTGGATTTGTAGGGGTAGACCACGTAGATAAGCTGATTGTCGTAAGCTTTC |
| | GCGGAACAACATCCGTTCGTAATTGGATCGCTGACCTGCTTTTCTTTCAGGTAAGTTGTGAC |
| | CTTACACCAGGCTGTTTGGCCCATGCCGGGTTTTACGCAAGCTGGGAGGAGGTCAAATCGCG |
| | CGTTTTAGACGCAGTCAAAAAGGCAAAAGCAGCGAACCCTTCATACCGCGTAGTTGCTACTG |
| | GATATAGTCTTGGCGGTGCAATCGCTACGTTAGCCGTTGCCTATATTCGCCAGCAAGGCCAT |
| | GCCGCGGATCTTTATACCTACGGTGGTCCGCGCGTTGGAAACCTGGCATTGGCCGAATTCAT |
| | TACTAATCAGCCCGGCGGGGAGTATCGCATTACGCACAAAGATGATCCTGTGCCCCGCCTTC |
| | CACCTTTAATTCTGAATTATCGCCACACGTCCCCAGAATATTGGATTGACCCGGGTAACGAC |
| | CGCACCGTCTCTTTAGACGAAGTCCAGTACTGTCCGGGATACTCGAATACCGACTGCAATGG |
| | AGGTACTGACGGATTAAATTTAGATAGTCACGGATTCTATTTTCAGTCTTTTATCGGCTGTG |
| | GACCAGGGTCGACTCCATGGCGTCGTGCATCAGATATTTCGGACAAGGAGCTGGAAGACCGT |
| | GTCAATTTGTTCGCGGAGCTGGACCGTCAACTTGCGAAGTCCCTTCACGAAGCGGGGGAGCA |
| | A |

### EXAMPLES

The examples set forth below illustrate certain embodiments and do not limit the technology. Certain examples set forth below utilize standard recombinant DNA, membrane vesicle/liposome preparation and other biotechnology protocols known in the art.

### Example 1

### Expression and Purification of Polypeptide Q2

DNA (SEQ ID NO:3), which is a codon optimized sequence encoding Polypeptide Q2 (SEQ ID NO:1), was introduced into a plasmid for recombinant expression of Polypeptide Q2, followed by purification of the recombinant expressed Polypeptide Q2, as follows:

A. Transformation of Rosetta Cells with Polypeptide Q2 DNA

A 50 μl aliquot of Rosetta (DE3) pLysS cells was transformed with 50 ng of pET28b(+)-Polypeptide Q2 plasmid DNA (a plasmid construct containing the sequence of polynucleotides, SEQ ID NO:3, which encodes Polypeptide Q2) by heating on a mini heat block at 42° C. for 90 secs. The transformation solution was chilled on ice for 2 mins. A volume of 445 μl of SOC media was added to the cell suspension, which was then incubated at 37° C. for 1 hour on a tabletop incubator with shaking at 225 rpm. The cells were pelleted by spinning at 6,000 rpm for 6 minutes on a tabletop centrifuge. A volume of 300 μl of supernatant was extracted and discarded, and the cell pellet was then resuspended in the remaining 200 μl of media. The cell suspension (200 μl) was added to the center of a LB-agar plate containing a final concentration of 30 μg/ml kanamycin and 34 μg/mIchloramphenicol. The cell suspension was spread evenly on the plate using a sterilized glass cell spreader. The plate was allowed to dry for 10 minutes and then incubated overnight at 37° C.

B. Picking Colonies and Establishing An Overnight Culture

3 medium sized colonies were picked with a sterile 200 μl pipette tip and delivered to 100 ml of LB broth containing a final concentration of 30 μg/mIkanamycin and 34 μg/ml chloramphenicol in a 500 ml Erlenmeyer flask. The overnight culture was incubated at 37° C. with shaking at 225 rpm for ≥16 hours.

C. Back Dilution of Overnight Culture and Induction of Recombinant Gene Expression

A volume of 20 ml of the saturated overnight culture was back diluted into 1 L Erlenmeyer flasks containing 500 ml fresh LB broth dosed with 30 μg/mIkanamycin and 34 μg/ml chloramphenicol. A total of 8 flasks (4 liters) were prepared from 100 ml of overnight culture (10 ml of overnight culture/500 ml media). The flasks were incubated at 37° C. shaking at 225 rpm until the absorbance at 600 nm (OD₆₀₀) was between 0.6 and 1.0 (approximately 2.5-3.5 hrs). IPTG was added to the overnight cultures in the 8 Erlenmeyer flasks to a final concentration of 1 mM. The flasks were transferred to a platform shaker at room temperature for shaking overnight (≥16 hrs) at 225 rpm. After induction, the absorbance at 600 nm of 1 ml of the cells was measured on a UV-Vis spectrophotometer and recorded. On average, cell cultures expressing Polypeptide Q2 are expected to display around a 60-65% reduction in OD₆₀₀.

D. Purification of 6×His-Polypeptide Q2 from Rosetta Cell Culture Supernatant

4 L of cell suspension was aliquoted evenly into six 500 ml centrifuge bottles (250 ml per bottle) and centrifuged at 4,000×g for 20 mins. Centrifugation was repeated until all 4 L were spun down and cells were separated from the supernatant. The cells were discarded, and the supernatant was pooled in 1 L aliquots in four clean 1 L bottles. A total volume of 10 ml of 3 M imidazole was added to each 1 L bottle to bring the final imidazole concentration to 30 mM. Bed volumes (15 ml) of Ni-NTA resin were poured into two 3.175 cm×53.34 cm glass columns and 5 ml of Ni-NTA is poured into two 3.175 cm×33.02 cm glass columns. All columns were fitted with stopcocks and pre-equilibrated in RODI water. A volume of ˜250 ml of supernatant was poured over the two ml Ni-NTA columns. The smaller 5 ml columns were positioned in tandem under the larger 15 ml columns. All pre-eluate solutions run over 15-ml and 5-ml resin beds were collected and pooled in a 4 L plastic beaker. When the drip rate dropped to <1 drop/3 seconds, the flow was halted using the stopcock. The resin bed was mixed by pipetting up and down using a pipette-gun fitted with a 25 ml serological pipette.

After the supernatant was loaded onto the columns, the resin beds were washed in 10 column volumes (CV) of wash buffer (20 mM sodium phosphate pH: 7.4, 300 mM NaCl, 30 mM imidazole) separately. After washing, the bound protein was eluted by running 1 CV of elution buffer (20mM sodium phosphate pH: 7.4, 300 mM NaCl, 250 mM imidazole) over the resin bed. The elution step was repeated once for each column (a total of 8 elutions). The eluate from each column was collected in a separate 50 ml conical tube. A 30 μl sample was taken from each eluate, added to 10 μl of 4× reducing SDS-sample buffer and boiled at 95° C. for 10min. on a mini heat block. The samples were cooled to room temperature and loaded onto a 4-20% gradient tris-glycine SDS-PAGE gel. One lane of each gel was loaded with 5 μl of a molecular weight standard diluted in 25 μl of 1× SDS-sample buffer (PageRuler Plus, Thermo Fisher Scientific, Inc., Waltham, Mass.) for molecular weight (MW) estimation. The gels were run at a constant 200V for approximately 45 min. and stained with AcquaStain protein dye (Bulldog Bio, Inc., Portsmouth, N.H.) overnight. The most intense band in the Ni-NTA eluates was consistent with the size of 6×His-Polypeptide Q2.

E. Desalting/buffer Exchanging Polypeptide Q2 Eluates into Sodium Acetate, pH: 6.0 for Differential Protein Precipitation and Further Purification of Polypeptide Q2

Eluates were pooled and concentrated to a volume of 8 ml using a 10,000 Da MWCO centrifugal concentrator. The total volume of the Ni-NTA eluates was subsequently loaded 12 ml at a time into the concentrator and centrifuged at 6,000×g for 20-30-minute intervals until the desired volume was reached. The filtrate was discarded as the retentate contains recombinant Polypeptide Q2. All 8 ml of concentrated Ni-NTA eluate was loaded into a 10,000 Da MWCO dialysis cassette and dialyzed against 4 L of 25 mM sodium acetate, pH: 6.0 (no salt). The initial 4 L dialysis was conducted for 3 hours at 4° C. and the dialysis buffer was changed. The second 4 L dialysis was performed overnight at 4° C. (˜16 hours).

Following dialysis, the protein solution was visibly turbid, indicating protein precipitation/aggregation. The dialysis buffer was changed 2 additional times with incubation periods of ˜3 hours between changes. The turbidity of the solution remained constant through the remainder of the dialysis process. The protein solution was extracted from the interior of the dialysis cassette using a needle and syringe, transferred to a 50 ml conical tube and centrifuged at 29,097×g for 20minutes. The supernatant was recovered, and the protein pellet was resuspended in 400 μl of 25 mM sodium acetate, pH: 6.0 containing 50 mM NaCl. A 30 μl sample of the supernatant and the reconstituted pellet was combined with 10 μl of 4× reducing SDS-sample buffer respectively.

The samples were boiled at 95° C. for 10mins. on a mini heat block and 30 μl were run on a 4-20% tris-glycine gradient SDS-PAGE gel along with PageRuler Plus pre-stained ladder (as described above) at constant 200V for approximately 45 mins. until the dye front reached the bottom of the gel. The gel confirmed that only the desired kDa band consistent with recombinant Polypeptide Q2 remained in the supernatant; all contaminant bands were partitioned into the insoluble pellet fraction. Another round on a centrifugal concentrator was used to decrease the overall volume of the soluble Polypeptide Q2 sample to ˜1 ml. A volume of 25 μl of 2 M NaCl was added to the concentrated Polypeptide Q2 (for a final concentration of 50 mM NaCl) sample to stabilize the protein and prevent further aggregation.

F. Estimation of 6×His-Polypeptide Q2 Protein Concentration and Dilution for Listeria Monocytogenes Biofilm Removal Assay

A combination of absorbance at 280 nm coupled with the predicted extinction coefficient and MW of 6×His-Polypeptide Q2, and the use of a 660 nm assay reagent kit (Thermo Fisher Scientific, Inc., Waltham, Mass.) estimateed the concentration of 6×His-Polypeptide Q2 between 150 and 300 μg/ml. The biofilm removing ability of recombinant 6×His-Polypeptide Q2 was tested in an in vitro assay against Listeria biofilms. A 100 μl volume of the 6×His-Polypeptide Q2 sample was diluted 10-fold in 1-ml of enzyme diluent (25 mM Tris-HCl, pH: 7.5, 2.5 mM CaCl₂, 2.75 mM MgCl₂). Both undiluted (in 25 mM sodium acetate, pH: 6.0, 50 mM NaCl) and diluted 6×His-Polypeptide Q2 samples were used in the Listeria biofilm removal assay. Enzyme diluent and 25 mM sodium acetate, pH: 6.0 with 50 mM NaCl were provided as negative controls.

### Example 2

### Biofilm Removal Activity of Polypeptide Q2

A modified version of the method presented by Djordjevic, Wiedmann, and McLandsborough (Appl. Environ. Microbiol., 68(6):2950-2958, 2002) was used for measuring biofilm formation. An isolated Listeria monocytogenes colony was selected and transferred to 10 mL of liquid growth media at 37° C. overnight. The overnight culture (0.1 mL) was transferred to 10 mL of fresh growth media. The culture was vortexed and 100 μL volumes were transferred to eight columns of 6 PVC microtiter plate wells previously rinsed with 70% ethanol and air dried in a biological safety hood for 40 min. Each plate was loaded in triplicate, covered, and incubated at the appropriate temperature for 48 hrs.

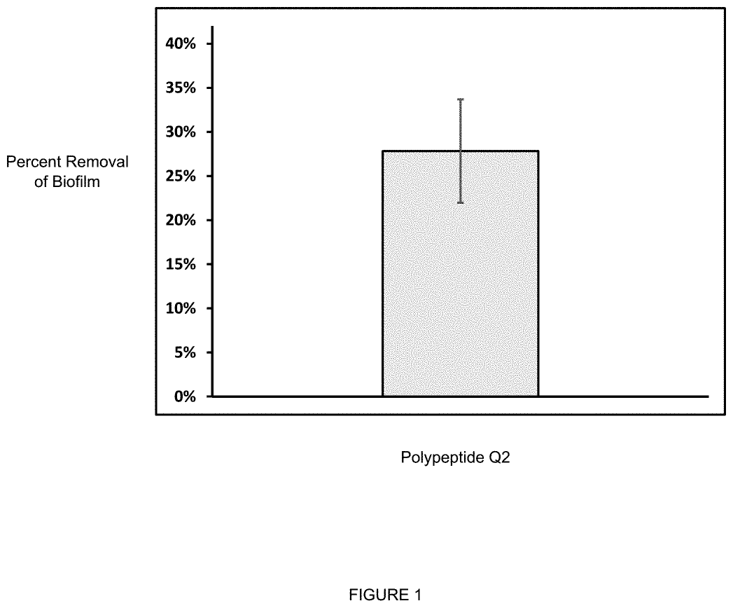

Following incubation, the media was removed. Each well was washed 5 times with 100 μL of sterile deionized water and allowed to air dry for 40 mins. Negative control, undiluted 6×His-Polypeptide Q2 samples or diluted 6×His-Polypeptide Q2 samples as described in Example 1, part F (150 μL) were added to each well and incubated at the desired temperature for either two or four hours. The samples were removed. Each well was washed 5 times with 100 μL of sterile deionized water and allowed to air dry for 40 min. Once dry, the wells were stained with a 0.1% crystal violet solution (150 μL ) for 45 min. Each well was washed 5 times with 100 μL of deionized water and allowed to air dry for 40 mins. Stained wells were de-stained for 1 hour with 200 μL of 95% ethanol. Samples (100 μL) were taken from each well and transferred to a new PVC microtiter plate and the absorbance was read at 570 nm using a SpectraMax Pro 384 96-well plate reader. FIG. 1 shows efficacy of biofilm removal by Polypeptide Q2 as measured in terms of percent removal of biofilm (y-axis).

Non-limiting Embodiments

Listed hereafter are non-limiting examples of certain embodiments of the technology.

A1. A pharmaceutical composition, comprising:

(a) one or more polypeptides comprising the sequence of amino acids set forth in SEQ ID NO:1 and/or comprising a consecutive sequence of amino acids that is 90% or more identical to a corresponding consecutive sequence of amino acids set forth in SEQ ID NO:1; and (b) a pharmaceutically acceptable excipient.

A2. The pharmaceutical composition of embodiment A1, wherein the composition exhibits biofilm removal activity, bactericidal activity, or both biofilm removal activity and bactericidal activity, or antiviral activity.

A3. A pharmaceutical composition for removing biofilm in or on a subject, comprising:

(a) one or more polypeptides exhibiting biofilm removal activity and selected from among (i) a polypeptide of SEQ ID NO:1, (ii) a portion of the polypeptide of SEQ ID NO:1, wherein the portion exhibits biofilm removal activity, and/or (iii) a polypeptide comprising a consecutive sequence of amino acids that is 90% or more identical to a consecutive sequence of amino acids set forth in SEQ ID NO:1 and exhibits biofilm removal activity; and

(b) a pharmaceutically acceptable excipient.

A4. The pharmaceutical composition of any one of embodiments A1-A3, wherein the polypeptide(s) exhibit biofilm removal activity.

A5. The pharmaceutical composition of any one of embodiments A1-A4, wherein the polypeptide(s) exhibit biofilm removal activity and the polypeptide(s) is/are in an amount sufficient to remove at least 20% of the biofilm associated with mucosa, a wound or a sore in or on a subject.

A6. The pharmaceutical composition of any one of embodiments A1-A5, wherein the composition, the polypeptide(s), or both the composition and the polypeptides do not exhibit bactericidal activity.

A7. The pharmaceutical composition of any one of embodiments A1-A6, comprising a single polypeptide.

A8. The pharmaceutical composition of embodiment A7, consisting essentially of a single polypeptide.

A9. The pharmaceutical composition of embodiment A7, consisting of a single polypeptide.

A10. The pharmaceutical composition of any one of embodiments A1-A6, comprising a plurality of polypeptides.

A11. The pharmaceutical composition of any one of embodiments A1-A7 and A10, wherein a polypeptide exhibiting biofilm removal activity comprises the polypeptide of SEQ ID NO:1.

A12. The pharmaceutical composition of embodiment A8 or A9, wherein the polypeptide exhibiting biofilm removal activity comprises the polypeptide of SEQ ID NO:1.

A13. The pharmaceutical composition of embodiment A8 or A9, wherein the polypeptide exhibiting biofilm removal activity consists essentially of the polypeptide of SEQ ID NO:1.

A14. The pharmaceutical composition of embodiment A8 or A9, wherein the polypeptide exhibiting biofilm removal activity consists of the polypeptide of SEQ ID NO:1.

A15. The pharmaceutical composition of any one of embodiments A2-A14, wherein the biofilm is associated with mucosa, a wound or a sore in or on a subject.

A16. The pharmaceutical composition of embodiment A15, wherein the biofilm is 15associated with a wound.

A17. The pharmaceutical composition of embodiment A16, wherein the wound is a chronic wound.

A18. The pharmaceutical composition of embodiment A17, wherein the subject has a disease or condition selected from among wound infection, an ischemic condition, a 20metabolic condition, immunosuppression and radiation.

A19. The pharmaceutical composition of embodiment A18, wherein the subject has a metabolic condition.

A20. The pharmaceutical composition of embodiment A19, wherein the metabolic condition is diabetes mellitus.

A21. The pharmaceutical composition of embodiment A15, wherein the biofilm is associated with mucosa.

A22. The pharmaceutical composition of embodiment A21, wherein the subject has cystic fibrosis or tuberculosis.

A23. The pharmaceutical composition of embodiment Al or A2 that is formulated for treatment of a subject with COVID-19.

A24. The pharmaceutical composition of any one of embodiments A1-A23, which is a gel, ointment, liquid, suspension, aerosol, powder or lyophile.

A25. The pharmaceutical composition of any one of embodiments Al -A24, formulated for single dosage administration.

A26. The pharmaceutical composition of any one of embodiments Al -A24, formulated for multiple dosage administration.

A27. The pharmaceutical composition of any one of embodiments Al -A26 that is a sustained release formulation.

A28. The pharmaceutical composition of any one of embodiments Al -A27, further comprising an additional therapeutically active agent.

A29. The pharmaceutical composition of embodiment A28, wherein the additional therapeutically active agent is selected from among one or more of: an analgesic agent, an anti-inflammatory agent and an antimicrobial agent.

A30. The pharmaceutical composition of embodiment A29, wherein the additional therapeutically active agent is an antimicrobial agent.

A31. The pharmaceutical composition of embodiment A30, wherein the antimicrobial agent is an antibiotic.

A32. The pharmaceutical composition of any one of embodiments A28-A31, wherein the polypeptide(s) and the additional therapeutically active agent are co-formulated.

A33. The pharmaceutical composition of any one of embodiments A28-A31, wherein the polypeptide(s) and the additional therapeutically active agent each are independently formulated.

A34. A composition for disinfecting a surface, comprising one or more polypeptides exhibiting biofilm removal activity and selected from among (i) a polypeptide of SEQ ID NO:1, (ii) a portion of the polypeptide of SEQ ID NO:1, wherein the portion exhibits biofilm removal activity, and/or (iii) a polypeptide comprising a consecutive sequence of amino acids that is 90% or more identical to a consecutive sequence of amino acids set forth in SEQ ID NO:1 and exhibits biofilm removal activity.

A35. The composition of embodiment A34, wherein the surface is a hard surface, a soft surface or a porous surface.

A36. The composition of embodiment A35, wherein the surface is a porous surface and the porous surface is selected from among skin, keratin and internal organs.

A37. The composition of any one of embodiments A34-A36, wherein disinfection comprises one or more of biofilm removal activity, bacteriostatic activity, bactericidal activity, and antiviral activity.

A38. The composition of embodiment A37, wherein disinfection consists of biofilm removal activity.

A39. The composition of embodiment A37, wherein disinfection comprises biofilm removal activity and one or more of bacteriostatic activity and bactericidal activity.

A40. The composition of embodiment A34, wherein disinfection comprises reduction in the amount of SARS-CoV-2 virus.

B1. A method of treating a disease or condition in a subject comprising administering, to a subject in need thereof, a therapeutically effective amount of the pharmaceutical composition of any one of embodiments A1-A33.

B1′. The method of embodiment B1, wherein the disease or condition is associated with a wound, sore or infection.

B2. The method of embodiment B1 or B1′, wherein the subject has a disease or condition selected from among ulcers, a wound infection, an ischemic condition, a metabolic condition, immunosuppression, radiation, cystic fibrosis, tuberculosis, and COVID-19.

B3. The method of embodiment B2, wherein the disease or condition is a metabolic condition.

B4. The method of embodiment B3, wherein the metabolic condition is diabetes mellitus.

B5. The method of any one of embodiments B1-B4, wherein there is biofilm associated with the disease or condition.

B6. The method of embodiment B5, wherein the biofilm associated with the disease or condition is reduced by at least 20%.

C1. A method of treating a disease or condition in a subject comprising administering, to a subject in need thereof: (a) a therapeutically effective amount of the pharmaceutical composition of any one of embodiments A28-A33, or (b) a therapeutically effective amount of the pharmaceutical composition of any one of embodiments A1-A27, and an additional therapeutically active agent.

C1′. The method of embodiment C1, wherein the disease or condition is associated with a wound, sore or infection.

C2. The method of embodiment C1 or C1′, wherein the additional therapeutically active agent is selected from among one or more of: an analgesic agent, an anti-inflammatory agent and an antimicrobial agent.

C3. The method of embodiment C1 or C2,wherein the subject has a disease or condition selected from among wound infection, an ischemic condition, a metabolic condition, immunosuppression, radiation, cystic fibrosis, tuberculosis, and COVID-19.

C4. The method of embodiment C3, wherein the disease or condition is a metabolic condition.

C5. The method of embodiment C4, wherein the metabolic condition is diabetes mellitus.

C6. The method of any one of embodiments C1-C5, wherein the therapeutically active agent is an antimicrobial agent.

C7. The method of embodiment C6, wherein the antimicrobial agent is an antibiotic.

C8. The method of any one of embodiments C1-C7, wherein the pharmaceutical composition and the additional therapeutically active agent are administered simultaneously, sequentially or intermittently.

C9. The method of any one of embodiments C1-C8, wherein the pharmaceutical composition is administered prior to administering the therapeutically active agent.

C10. The method of any one of embodiments C1-09, wherein there is biofilm associated with the disease or condition.

C11. The method of embodiment C10, wherein the biofilm associated with the disease or condition is reduced by at least 20%.

D1. A device, comprising the pharmaceutical composition of any one of embodiments A1-A33.

D2. The device of embodiment D1 that is a wound dressing, a topical patch, syringe, an inhaler, a dosage cup, a dropper, a pump, a spray bottle, an aerosol container or an applicator for administering the pharmaceutical composition.

D3. The device of embodiment D2 that is a pump for irrigation of a wound or sore with the pharmaceutical composition.

D4. The device of embodiment D2 that is a spray bottle or aerosol container for coating a wound or sore with the pharmaceutical composition.

E1. A kit, comprising a pharmaceutical composition of any one of embodiments A1-A33 and a device for administration of the composition.

10E2. The kit of embodiment E1, wherein the pharmaceutical composition is contained in the device for administration.

E3. The kit of embodiment E1, wherein the pharmaceutical composition is present as a separate component that is distinct from the device.

E4. The kit of any one of embodiments E1-E3, wherein the device is a dressing, a 15topical patch, a pump, a spray bottle, an aerosol container, a syringe, an inhaler, a dosage cup, a dropper, or an applicator.

F1. A polynucleotide encoding a polypeptide comprising the sequence of amino acids set forth in SEQ ID NO:1 and/or comprising a consecutive sequence of amino acids that is 90% or more identical to a corresponding consecutive sequence of amino acids set forth in SEQ ID NO:1, wherein the polynucleotide comprises: (i) a polynucleotide of SEQ ID NO:2, (ii) a polynucleotide comprising a consecutive sequence of amino acids that is 80% or more identical to a consecutive sequence of amino acids set forth in SEQ ID NO:2, (iii) a polynucleotide of SEQ ID NO:3, or (iv) a polynucleotide that encodes a consecutive sequence of amino acids that is 80% or more identical to a consecutive sequence of amino acids set forth in SEQ ID NO:1.

F2. The polynucleotide of embodiment F1, wherein the polypeptide encoded by the polynucleotide exhibits biofilm removal activity.

F3. A vector, comprising the polynucleotide of embodiment F1 or F2. F4. The vector of embodiment F3 that is an expression vector. F5. The vector of embodiment F3 or F4, comprising the sequence of nucleotides set forth in SEQ ID NO:3.

G1. A method of disinfecting a surface comprising applying, to the surface, the composition of embodiment A34.

G2. The method of embodiment G1, wherein the surface is a hard surface, a soft surface or a porous surface.

5 G3. The method of embodiment G2, wherein the surface is a porous surface and the porous surface is selected from among skin, keratin, mucous membranes, and internal organs.

G4. The method of any one of embodiments G1-G3, wherein disinfection comprises one or more of biofilm removal activity, bacteriostatic activity, bactericidal activity, and 10antiviral activity.

G5. The method of embodiment G4, wherein disinfection consists of biofilm removal activity.

G6. The method of embodiment G4, wherein disinfection comprises biofilm removal activity and one or more of bacteriostatic activity and bactericidal activity.

H1. An isolated, recombinant or synthetically produced polypeptide comprising a consecutive sequence of amino acids that is 80% or more identical, or 90% or more identical to a corresponding consecutive sequence of amino acids set forth in SEQ ID NO:1.

H2. The polypeptide of embodiment H1, comprising a sequence of amino acids that is 80% or more identical, or 90% or more identical to the sequence of amino acids set forth in SEQ ID NO: 1.

H3. An isolated, recombinant, or synthetically produced polypeptide comprising a portion of a sequence of amino acids that is 80% or more identical, 90% or more identical, or 100% identical to the sequence of amino acids set forth in SEQ ID NO: 1, wherein the portion exhibits biofilm removal activity and/or enzyme activity.

H4. The polypeptide of any one of embodiments H1-H3, wherein the polypeptide exhibits enzyme activity.

H5. The polypeptide of embodiment H4, wherein the polypeptide exhibits esterase activity.

H6. The polypeptide of embodiment H4, wherein the polypeptide exhibits cutinase activity.

H7. The polypeptide of any one of embodiments H4-H6, wherein the enzyme activity is thermally stable.

H8. The polypeptide of any one of embodiments H1-H7, wherein the polypeptide exhibits biofilm removal activity, microbicidal activity, and/or microbiostatic activity.

H9. The polypeptide of any one of embodiments H1-H7, wherein the polypeptide exhibits biofilm removal activity, bactericidal activity and/or bacteriostatic activity.

H10. The polypeptide of any one of embodiments H1-H7, wherein the polypeptide exhibits biofilm removal activity but does not exhibit bactericidal activity and/or bacteriostatic activity.

I1. An isolated or recombinant polynucleotide encoding the polypeptide of any one of embodiments H1-H10.

I2. The polynucleotide of embodiment I1, comprising a consecutive sequence of nucleotides that is 80% or more identical, or 90% or more identical to a corresponding consecutive sequence of nucleotides set forth in SEQ ID NO: 2 or SEQ ID NO: 3.

3. The polynucleotide of embodiment I1, comprising (i) a sequence of nucleotides that is 80% or more identical, or 90% or more identical to the sequence of nucleotides set forth in SEQ ID NO:2, (ii) a sequence of nucleotides that is 80% or more identical, or 90% or more identical to the sequence of nucleotides set forth in SEQ ID NO:3, (iii) the sequence of nucleotides set forth in SEQ ID NO:2, or (iv) the sequence of nucleotides set forth in SEQ ID NO:3.

I4. The polynucleotide of any one of embodiments I1-I3, wherein the polypeptide encoded by the polynucleotide exhibits enzyme activity.

I5. The polynucleotide of embodiment I4, wherein the enzyme activity is thermally stable.

I6. The polynucleotide of any one of embodiments I1-I5, wherein the polypeptide encoded by the polynucleotide exhibits biofilm removal activity, microbicidal activity, and/or microbiostatic activity.

J1. A vector, comprising the polynucleotide of any one of embodiments I1-I6.

J2. The vector of embodiment J1 that is an expression vector.

J3. The vector of embodiment J1 or embodiment J2, comprising the sequence of nucleotides set forth in SEQ ID NO:3.

K1. A composition, comprising one or more polypeptides of embodiments H1-H10.

K2. The composition of embodiment K1, wherein one or more polypeptides comprise a sequence of amino acids that is 80% or more identical, or 90% or more identical, or 100% identical to the sequence of amino acids set forth in SEQ ID NO: 1.

K3. The composition of embodiment K1 or embodiment K2, comprising about 0.1% to about 10% weight/volume of the one or more polypeptides.

K4. The composition of embodiment K1 or embodiment K2, wherein the amount of the one or more polypeptides is effective to reduce the amount of a biofilm.

K5. The composition of embodiment K4, wherein the amount of the one or more polypeptides is effective to reduce the amount of a biofilm by about 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more.

K6. The composition of any one of embodiments K1-K5, comprising a plurality of polypeptides selected from the polypeptides of embodiments H1-H10.

K7. The composition of any one of embodiments K1-K5, comprising a single polypeptide of any one of embodiments H1-H10.

K8. The composition of embodiment K7, consisting essentially of a single polypeptide of 20any one of embodiments H1-H10.

K9. The composition of embodiment K7, consisting of a single polypeptide of any one of embodiments H1-H10.

K10. The composition of any one of embodiments K1-K7, wherein one or more, or all, of the one or more polypeptides of embodiments H1-H10exhibits enzyme activity and the 25composition does not comprise any other polypeptide that exhibits enzyme activity.

K11. The composition of any one of embodiments K1-K10, wherein the composition further comprises an excipient.

K12. The composition of embodiment K11, wherein the excipient comprises a protectant, surfactant, buffer, and/or bulking agent.

K13. The composition of embodiment K11, wherein the excipient comprises a cryoprotectant and/or lyoprotectant.

K14. The composition of embodiment K11, wherein the excipient is a pharmaceutical excipient.

5 K15. The composition of any one of embodiments K1-K14, wherein the composition is in the form of a liquid, solid, semi-solid or mixture of a liquid, solid and/or semi-solid.

K16. The composition of any one of embodiments K1-K14, wherein the composition is in the form of a solution, dispersion, suspension, emulsion, or colloid.

K17. The composition of any one of embodiments K1-K14, wherein the composition is in the form of a cream, lotion, emulsion, oil, butter, paste, balm, stick, foam, gel, serum, ointment, mousse, powder, semi-solid formulation, patch, spray or aerosol.

K18. The composition of any one of embodiments K1-K14, wherein the composition is a lyophilizate.

K19. The composition of any one of embodiments K1-K18, wherein the composition is macroscopically homogeneous or macroscopically heterogeneous.

K20. The composition of any one of embodiments K1-K19, wherein the composition is formulated for topical administration to an animal.

K21. The composition of any one of embodiments K1-K20, containing less than 30% by weight of fungal cell lysate material.

K22. The composition of any one of embodiments K1-K20, containing less than 5% by weight of fungal cell lysate material.

K23. The composition of any one of embodiments K1-K20, containing less than 1% by weight of fungal cell lysate material.

K24. The composition of any one of embodiments K1-K20, containing essentially no fungal cell lysate material.

K25. The composition of any one of embodiments K21- K24, wherein the fungal cell lysate material is selected from 16S rRNA and the ITS1 region of the rRNA cistron.

K26. The composition of any one of embodiments K21- K24, wherein the fungal cell lysate material is from the TM-417 fungus.

K27. The composition of any one of embodiments K1-K20, containing less than 30% by weight and greater than 0% by weight of bacterial cell lysate material.

K28. The composition of embodiment K27, wherein the bacterial cell lysate material is from E. coli.

K29. The composition of embodiment K28, wherein the E. coli is a strain selected from BL21, Rosetta-gami, and Shuffle T7 strains.

K30. The composition of embodiment K29, wherein the E. coli strain is BL21.

K31. The composition of any one of embodiments K1-K30, further comprising an antimicrobial agent.

K32. The composition of any one of embodiments K1-K30, further comprising one or more of an antibacterial agent, an antifungal agent and an antiviral agent.

K33. The composition of any one of embodiments K1-K30, further comprising an antibiotic.

L1. The composition of any one of embodiments K1-K33, wherein the composition is a pharmaceutical composition.

L2. The pharmaceutical composition of embodiment L1, wherein the composition exhibits biofilm removal activity, microbicidal activity, and/or microbiostatic activity.

L3. The pharmaceutical composition of embodiment L1 or embodiment L2, wherein the one or more polypeptides of embodiments H1 -H10exhibit biofilm removal activity.

L4. The pharmaceutical composition of embodiment L3, wherein a polypeptide that exhibits biofilm removal activity comprises the polypeptide of SEQ ID NO:1.

L5. The pharmaceutical composition of embodiment L3, wherein a polypeptide that exhibits biofilm removal activity consists essentially of the polypeptide of SEQ ID NO:1.

L6. The pharmaceutical composition of embodiment L3, wherein a polypeptide that exhibits biofilm removal activity consists of the polypeptide of SEQ ID NO:1.

L7. The pharmaceutical composition of any one of embodiments L1-L6, wherein the one or more polypeptides of embodiments H1 -H1 0 exhibit biofilm removal activity and the polypeptide(s) is/are in an amount sufficient to remove at least 20% of the biofilm associated with a tissue of a subject.

L8. The pharmaceutical composition of any one of embodiments L1-L7, wherein the composition, the one or more polypeptides of embodiments H1-H10, or both the composition and the polypeptide(s) do not exhibit microbicidal activity.

L9. The pharmaceutical composition of any one of embodiments L1-L7, wherein the composition, the one or more polypeptides of embodiments H1-H10, or both the composition and the polypeptide(s) do not exhibit bactericidal activity.

L10. The pharmaceutical composition of any one of embodiments L1-L9, comprising a plurality of polypeptides of embodiments H1-H10.

L11. The pharmaceutical composition of any one of embodiments L1-L9, comprising a single polypeptide of any one of embodiments H1-H10.

L12. The pharmaceutical composition of embodiment L11, consisting essentially of a single polypeptide of any one of embodiments H1-H10.

L13. The pharmaceutical composition of embodiment L12, consisting of a single polypeptide of any one of embodiments H1-H10.

L14. The pharmaceutical composition of any one of embodiments L1-L13 for treating a disease or condition associated with a microbial infection and/or susceptible to microbial infection.

L15. The pharmaceutical composition of embodiment L14, wherein the disease or condition comprises tissue injury or damage.

L16. The pharmaceutical composition of embodiment L15, wherein the tissue is epithelial or connective tissue.

L17. The pharmaceutical composition of embodiment L15, wherein the tissue is a membrane.

L18. The pharmaceutical composition of embodiment L17, wherein the membrane is a cutaneous membrane, mucous membrane, serous membrane or synovial membrane.

L19. The pharmaceutical composition of embodiment L15, wherein the tissue injury or damage comprises a sore, wound, burn, a skin ulcer, or an ulcer in the stomach mucosa or intestinal mucosa.

L20. A pharmaceutical composition of any one of embodiments L1-L13 for removing biofilm in or on a subject.

L21. The pharmaceutical composition of embodiment L20, wherein the biofilm is associated with tissue of a subject.

L22. The pharmaceutical composition of embodiment L21, wherein the tissue is epithelial or connective tissue.

L23. The pharmaceutical composition of embodiment L21, wherein the tissue is a membrane.

L24. The pharmaceutical composition of embodiment L23, wherein the membrane is a cutaneous membrane, mucous membrane, serous membrane or synovial membrane.

L25. The pharmaceutical composition of embodiment L20, wherein the biofilm is associated with a sore, wound, burn, a skin ulcer, or an ulcer in the stomach mucosa or intestinal mucosa.

L26. The pharmaceutical composition of embodiment L20, wherein the biofilm is associated with a wound.

L27. The pharmaceutical composition of embodiment L20, wherein the biofilm is associated with a chronic wound.

L28. The pharmaceutical composition of embodiment L27, wherein the subject has a disease or condition selected from among wound infection, an ischemic condition, a metabolic condition, immunosuppression and radiation exposure.

L29. The pharmaceutical composition of embodiment L25, wherein the subject has a metabolic condition.

L30. The pharmaceutical composition of embodiment L29, wherein the metabolic condition is diabetes mellitus.

L31. The pharmaceutical composition of embodiment L20, wherein the biofilm is associated with mucosa.

L32. The pharmaceutical composition of embodiment L31, wherein the subject has cystic fibrosis or tuberculosis.

L33. The pharmaceutical composition any one of embodiments L1-L13 that is formulated for treatment of a subject with COVID-19.

L34. The pharmaceutical composition of any one of embodiments L1-L33, comprising a pharmaceutically acceptable excipient.

L35. The pharmaceutical composition of any one of embodiments L1-L34, formulated for single dosage administration.

L36. The pharmaceutical composition of any one of embodiments L1-L34, formulated for multiple dosage administration.

L37. The pharmaceutical composition of any one of embodiments L1-L36 that is a sustained release formulation.

L38. The pharmaceutical composition of any one of embodiments L1-L37, wherein the composition is formulated for administration to an animal.

L39. The pharmaceutical composition of any one of embodiments L1-L37, wherein the composition is formulated for topical administration to an animal.

L40. The pharmaceutical composition of embodiment L38 or embodiment L39, wherein the animal is a human.

L41. The pharmaceutical composition of any one of embodiments L1-L40, further comprising an additional therapeutically active agent.

L42. The pharmaceutical composition of embodiment L41, wherein the additional therapeutically active agent comprises one or more of an analgesic agent, an anti-inflammatory agent and an antimicrobial agent.

L43. The pharmaceutical composition of embodiment L42, wherein the additional therapeutically active agent comprises an antimicrobial agent.

L44. The pharmaceutical composition of embodiment L43, wherein the additional therapeutically active agent comprises one or more of an antibacterial agent, an antifungal agent and an antiviral agent.

L45. The pharmaceutical composition of embodiment L43, wherein the additional therapeutically active agent comprises an antibiotic.

L46. The pharmaceutical composition of any one of embodiments L41-L45, wherein the one or more polypeptides of embodiments H1 -H10 and the additional therapeutically active agent are co-formulated.

L47. The pharmaceutical composition of any one of embodiments L41-L45, wherein the one or more polypeptides of embodiments H1 -H10 and the additional therapeutically active agent each are independently formulated.

M1. The composition of any one of embodiments K1-K33, wherein the composition is for treating a surface.

M2. The composition of embodiment Ml, wherein the composition exhibits biofilm removal activity, microbiostatic activity, and/or microbicidal activity.

M3. The composition of embodiment M1 or embodiment M2, wherein the one or more polypeptides of embodiments H1-H10 exhibit biofilm removal activity.

M4. The composition of embodiment M3, wherein a polypeptide that exhibits biofilm removal activity comprises the polypeptide of SEQ ID NO:1.

M5. The composition of embodiment M3, wherein a polypeptide that exhibits biofilm removal activity consists essentially of the polypeptide of SEQ ID NO:1.

M6. The composition of embodiment M3, wherein a polypeptide that exhibits biofilm removal activity consists of the polypeptide of SEQ ID NO:1.

M7. The composition of any of embodiments M1-M6, wherein treating comprises one or more of biofilm removal activity, microbiostatic, and/or microbicidal activity.

M8. The composition of any of embodiments M1-M6, wherein treating comprises one or more of biofilm removal activity, bacteriostatic activity, bactericidal activity, antifungal and antiviral activity.

M9. The composition of any of embodiments M1-M6, wherein treating comprises biofilm removal activity and one or more of bacteriostatic activity and bactericidal activity.

M10. The composition of any of embodiments M1-M6, wherein treating consists essentially of biofilm removal activity.

M11. The composition of any of embodiments M1-M6, wherein treating consists of biofilm removal activity.

M12. The composition of any of embodiments M1-M10, wherein treating comprises one or more of disinfection, antisepsis, sanitization and cleaning.

M13. The composition of any one of embodiments M1-M12, wherein the one or more polypeptides of embodiments H1-H10 exhibit biofilm removal activity and the polypeptide(s) is/are in an amount effective to reduce the amount of a biofilm associated with the surface by about 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more.

M14. The composition of embodiment M13, wherein the one or more polypeptides of embodiments H1-H10 is/are in an amount effective to reduce the amount of a biofilm associated with a surface by about 20% or more.

M15. The composition of any one of embodiments M1-M12, comprising about 0.1% to about 10% weight/volume of the one or more polypeptides of embodiments H1-H10.

M16. The composition of any one of embodiments M1-M14, wherein the composition, the one or more polypeptides of embodiments H1-H10, or both the composition and the polypeptide(s) do not exhibit microbicidal and/or microbiostatic activity.

M17. The composition of any one of embodiments M1-M14, wherein the composition, the one or more polypeptides of embodiments H1-H10, or both the composition and the polypeptide(s) do not exhibit bactericidal and/or bacteriostatic activity.

M18. The composition of any one of embodiments M1-M17, wherein treating comprises reduction in the amount of SARS-CoV-2 virus, herpes virus, and/or human papillomavirus.

M19. The composition of any one of embodiments M1-M18, wherein the surface is a hard surface, a soft surface or a porous surface.

M20. The composition of any one of embodiments M1-M19, wherein the surface is a biological surface.

M21. The composition of embodiment M20, wherein the surface is selected from among skin, keratin, hair, teeth, tissues and internal organs.

M22. The composition of embodiment M21, further comprising an antibiotic.

M23. The composition of embodiment M21, further comprising an antiseptic agent and/or antimicrobial agent.

M24. The composition of embodiment M23, wherein the antiseptic agent comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, a nucleoside analog, and plant extracts.

M25. The composition of any one of embodiments M1-M19, wherein the surface is an inanimate surface.

M26. The composition of embodiment M25, further comprising one or more of disinfecting agent, a sanitizing agent, a cleaning agent, and an antimicrobial agent.

M27. The composition of embodiment M25, further comprising one or more of bleach, chlorine compounds, alcohol, peroxide, phenol or derivative thereof, an aldehyde and a detergent.

N1. A combination, comprising:
(1) one or more polypeptides of embodiments H1-H10, and
(2) an agent, wherein the agent is:
   (a) a therapeutically active agent that does not comprise a polypeptide of embodiments H1-H10; or
   (b) a surface treatment agent that does not comprise a polypeptide of embodiments H1-H10;

wherein (1) and (2) are each in a separate composition or are in the same composition.

N2. The combination of embodiment N1, wherein the amount of (1) is effective to reduce the amount of a biofilm.

N3. The combination of embodiment N2, wherein the amount of (1) is effective to 20reduce the amount of a biofilm by about 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more.

N4. The combination of any one of embodiments N1-N3, wherein (2) comprises one or more of: an analgesic agent, an anti-inflammatory agent, a disinfecting agent, a sanitizing agent, a cleaning agent, an antiseptic agent and an antimicrobial agent.

N5. The combination of any one of embodiments N1-N3, wherein (2) comprises one or more of: a disinfecting agent, a sanitizing agent, a cleaning agent, an antiseptic agent, and an antimicrobial agent.

N6. The combination of embodiment N5, wherein (2) comprises one or more of: an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, plant extracts, bleach, chlorine compounds, alcohol, an aldehyde and a detergent.

N7. The combination of any one of embodiments N1-N3, wherein (2) comprises an antimicrobial agent.

N8. The combination of any one of embodiments N1-N3, wherein (2) comprises one or more of an antibacterial agent, an antifungal agent and an antiviral agent.

N9. The combination of embodiment N8, wherein (2) comprises a nucleoside analog and/or imidazole or derivative thereof.

N10. The combination of any one of embodiments N1-N3, wherein (2) comprises an antibiotic.

N11. The combination of any one of embodiments N1-N10, wherein the amount of (1) and (2) are such that when a surface is contacted with (1) and (2) the combination is effective to reduce the amount of one or more of viable bacteria, fungi and viruses, or reduce the growth of one or more of bacteria, fungi and viruses.

N12. The combination of any one of embodiments N1-N10, wherein the amount of (1) and (2) are such that when (1) and (2) are administered to a subject the combination is effective to reduce the amount of one or more of viable bacteria, fungi and viruses, or reduce the growth of one or more of bacteria, fungi and viruses in or on the subject.

N13. The combination of embodiment N10, wherein the amount of (1) and (2) are such that when (1) and (2) are administered to a subject the combination is effective to reduce the amount of viable bacteria, or reduce the growth of bacteria in or on the subject.

N14. The combination of any one of embodiments N1-N10, wherein the amount of (1) and (2) are such that when a surface is contacted with (1) and (2) the combination is effective to reduce a greater amount of one or more of viable bacteria, fungi and viruses on the surface than the amount of reduction of the one or more of viable bacteria, fungi and viruses on the surface when it is contacted with only one of (1) or (2).

N15. The combination of any one of embodiments N1-N10, wherein the amount of (1) and (2) are such that when (1) and (2) are administered to a subject the combination is effective to reduce a greater amount of one or more of viable bacteria, fungi and viruses in or on the subject than the amount of reduction of the one or more of viable bacteria, fungi and viruses in or on the subject when only one of (1) or (2) is administered to the subject.

N16. The combination of embodiment G10, wherein the amount of (1) and (2) are such that when (1) and (2) are administered to a subject the combination is effective to reduce a greater amount of viable bacteria in or on the subject than the amount of reduction of viable bacteria in or on the subject when only one of (1) or (2) is administered to the subject.

N17. The combination of any one of embodiments N1-N16, wherein (1) and/or (2) further comprises an excipient.

N18. The combination of embodiment N17, wherein the excipient comprises a protectant, surfactant, buffer, and/or bulking agent.

N19. The combination of embodiment N17, wherein the excipient comprises a cryoprotectant and/or lyoprotectant.

N20. The combination of embodiment N17, wherein the excipient is a pharmaceutical excipient.

N21. The combination of any one of embodiments N1-N20, wherein (1) and/or (2) is in the form of a liquid, solid, semi-solid or mixture of a liquid, solid and/or semi-solid.

N22. The combination of any one of embodiments N1-N20, wherein (1) and/or (2) is in the form of a solution, dispersion, suspension, emulsion, or colloid.

N23. The combination of any one of embodiments N1-N20, wherein (1) and/or (2) is in the form of a cream, lotion, emulsion, oil, butter, paste, balm, stick, foam, gel, serum, ointment, mousse, powder, semi-solid formulation, patch, spray or aerosol.

N24. The combination of any one of embodiments N1-N23, wherein (1) and/or (2) is formulated for topical administration to an animal.

N25. The combination of any one of embodiments N1-N24, wherein (1) and (2) are administered to a subject, or contacted with a surface, serially, sequentially, intermittently, concurrently or simultaneously.

N26. The combination of any one of embodiments N1-N25, wherein (1) and/or (2) is a lyophilizate.

O1. A method of removing biofilm and/or of treating a surface, comprising contacting the biofilm or surface with a composition of any one of embodiments K1-K33, a pharmaceutical composition of any one of embodiments L1-L47, the composition of any one of embodiments M1-M27, or a combination of any one of embodiments N1-N26.

O2. The method of embodiment O1, wherein the one or more polypeptides of embodiments H1-H10 of the composition, of the pharmaceutical composition, or of the combination exhibit biofilm removal activity and the polypeptide(s) is/are in an amount effective to reduce the amount of a biofilm associated with the surface by about 20%, 25% , 30%, 35%, 40%, 45%, 50%, 55%, 60%, 65%, 70%, 75%, 80%, 81%, 82%, 83%, 84%, 85%, 86%, 87%, 88%, 89%, 90%, 91 %, 92%, 93%, 94%, 95%, 96%, 97%, 98%, 99% or more.

O3. The method of embodiment O2, wherein the one or more polypeptides of embodiments H1-H10 of the composition, of the pharmaceutical composition, or of the combination is/are in an amount effective to reduce the amount of a biofilm associated with a surface by about 20% or more.

O4. The method of any one of embodiments O1-O3, wherein the composition, pharmaceutical composition, or combination comprises about 0.1% to about 10% weight/volume of the one or more polypeptides of embodiments H1-H10.

O5. The method of any one of embodiments O1-O4, wherein the one or more polypeptides of embodiments H1-H10 of the composition, of the pharmaceutical composition, or of the combination do not exhibit one or both of microbicidal activity and microbiostatic activity.

O6. The method of any one of embodiments O1-O4, wherein the one or more polypeptides of embodiments H1-H10 of the composition, of the pharmaceutical composition, or of the combination do not exhibit one or both of bactericidal activity and bacteriostatic activity.

O7. The method of any one of embodiments O1-O4, wherein the composition, pharmaceutical composition, or combination does not exhibit one or both of microbicidal activity and microbiostatic activity.

O8. The method of any one of embodiments O1-O4, wherein the composition, pharmaceutical composition, or combination does not exhibit one or both of bactericidal activity and bacteriostatic activity.

O9. The method of any one of embodiments O1-O6, wherein treating comprises one or more of biofilm removal activity, microbiostatic, and/or microbicidal activity.

O10. The method of any one of embodiments O1-O6, wherein treating comprises biofilm removal activity, and one or more of microbiostatic activity and microbicidal activity.

O11. The method of any one of embodiments O1-O6, wherein treating comprises biofilm removal activity, and one or more of bacteriostatic activity and bactericidal activity.

O12. The method of any one of embodiments O1-O6, wherein treating comprises biofilm removal activity.

O13. The method of any one of embodiments O1-O6, wherein treating consists essentially of biofilm removal activity.

O14. The method of any one of embodiments O1-O8, wherein treating consists of biofilm removal activity.

O15. The method of any one of embodiments O1-O6, wherein treating comprises reducing the amount of one or more of viable bacteria, fungi and viruses, and/or stopping the growth of one or more of bacteria, fungi and viruses.

O16. The method of any one of embodiments O1-O6, wherein treating comprises reducing the amount of one or more of viable SARS-CoV-2 virus, herpes virus and human papillomavirus or stopping the growth of one or more of viable SARS-CoV-2 virus, herpes virus and human papillomavirus.

O17. The method of any one of embodiments O1-O16, wherein treating comprises one or more of disinfection, antisepsis, sanitization and cleaning.

O18. The method of any one of embodiments O1-O6, wherein removing biofilm further comprises reducing the amount of viable microbes and/or reducing the growth of microbes.

O19. The method of embodiment O18, wherein the microbes comprise one or more of bacteria, fungi and viruses.

O20. The method of any one of embodiments O1-O6, wherein removing biofilm further comprises reducing the amount of viable SARS-CoV-2 virus, herpes virus, and/or human papillomavirus or stopping the growth of one or more of viable SARS-CoV-2 virus, herpes virus and human papillomavirus.

O21. The method of any one of embodiments O1-O17, wherein the surface is a hard surface, a soft surface or a porous surface.

O22. The method of any one of embodiments O1-O17 and O21, wherein the surface is a biological surface.

O23. The method of any one of embodiments O1-O6, O18 and O19, wherein the biofilm is in or on a biological substance.

O24. The method of any one of embodiments O1-O16 and O21-O23, wherein the surface or biological substance is selected from among skin, keratin, hair, teeth, tissues and internal organs.

O25. The method of embodiment O24, wherein the tissue is epithelial or connective tissue.

O26. The method of embodiment O25, wherein the tissue is a membrane.

O27. The method of embodiment O25, wherein the membrane is a cutaneous membrane, mucous membrane, serous membrane or synovial membrane.

O28. The method of any one of embodiments O25-O27, wherein the tissue is injured or damaged.

O29. The method of embodiment O28, wherein the tissue injury or damage comprises a sore, wound, burn, a skin ulcer, or an ulcer in the stomach mucosa or intestinal mucosa.

O30. The method of embodiment O29, wherein the wound is a chronic wound.

O31. The method of any one of embodiments O1-O17 and O21-O24, wherein the composition, pharmaceutical composition, or combination comprises an antibiotic.

O32. The method of embodiment O31, wherein the one or more polypeptides of embodiments H1-H10 of the composition, pharmaceutical composition, or combination and the antibiotic are contacted with the biofilm or surface serially, sequentially, intermittently, concurrently or simultaneously.

O33. The method of any one of embodiments O1-O17 and O21-O24, wherein the composition, pharmaceutical composition, or combination comprises an antiseptic agent and/or antimicrobial agent.

O34. The method of embodiment O33, wherein the one or more polypeptides of embodiments H1-H10 of the composition, pharmaceutical composition, or combination and the antiseptic agent and/or antimicrobial agent are contacted with the biofilm or surface serially, sequentially, intermittently, concurrently or simultaneously.

O35. The method of embodiment O33 or embodiment O34, wherein the antiseptic agent comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, a nucleoside analog, and plant extracts.

O36. The method of any one of embodiments O1-O17 and O21, wherein the surface is an inanimate surface.

O37. The method of any one of embodiments O1-O6 and O17-O20, wherein the biofilm is in or on an inanimate object.

O38. The method of embodiment O36 or embodiment O37, wherein the composition, pharmaceutical composition, or combination comprises one or more of a disinfecting agent, a sanitizing agent, a cleaning agent, and an antimicrobial agent.

O39. The method of embodiment O38, wherein the one or more polypeptides of embodiments H1-H10 of the composition, pharmaceutical composition, or combination and the one or more of a disinfecting agent, a sanitizing agent, a cleaning agent, and an antimicrobial agent is/are contacted with the biofilm or surface serially, sequentially, intermittently, concurrently or simultaneously.

O40. The method of embodiment O37 or embodiment O38, wherein the composition, pharmaceutical composition, or combination comprises one or more of bleach, a chlorine compound, alcohol, peroxide, a phenol or derivative thereof, an aldehyde and a 25detergent.

O41. The method of embodiment O40, wherein the one or more polypeptides of embodiments H1-H10 of the composition, pharmaceutical composition, or combination and the one or more of bleach, a chlorine compound, alcohol, peroxide, a phenol or derivative thereof, an aldehyde and a detergent is/are contacted with the biofilm or surface serially, sequentially, intermittently, concurrently or simultaneously.

P1. A method of treating a disease or condition of a subject comprising administering, to a subject in need thereof, a composition of any one of embodiments K1-K33, a pharmaceutical composition of any one of embodiments L1-L47, the composition of any one of embodiments M1-M27, or a combination of any one of embodiments N1-N26.

P2. The method of embodiment P1, wherein there is biofilm associated with the disease or condition.

P3. The method of embodiment P1 or embodiment P2, comprising administering a therapeutically effective amount of the composition, the pharmaceutical composition, or combination.

P4. The method of embodiment P1 or embodiment P2, comprising administering:
(a) (i) a therapeutically effective amount of the composition of any one of embodiments K1-K30and (ii) one or more of an antimicrobial agent, an antibacterial agent, an antifungal agent, an antiviral agent and antibiotic of any one of embodiments K31-K33;
(b) (i) a therapeutically effective amount of the pharmaceutical composition of any one of embodiments L1-L40 and (ii) an additional therapeutically active agent of any one of embodiments L41-L47;
(c) (i) a therapeutically effective amount of the composition of embodiment M20 or embodiment M21 and (ii) one or more of an antibiotic, an antiseptic agent, and an antimicrobial agent of any one of embodiments M22-M24; or
(d) (i) a therapeutically effective amount of one or more polypeptides of embodiments H1-H10 of the combination of embodiments N1-N3 and (ii) a therapeutically active agent or a surface treatment agent of any one of embodiments N4-N10.

P5. The method of any one of embodiments P1-P4, wherein the disease or condition is associated with a microbial infection and/or susceptible to microbial infection.

P6. The method of any one of embodiments P1-P5, wherein the disease or condition is associated with tissue injury or damage.

P7. The method of embodiment P6, wherein the tissue injury or damage comprises a sore, wound or burn.

P8. The method of embodiment P6, wherein the tissue injury or damage comprises an ulcer in the stomach mucosa or intestinal mucosa or a skin ulcer.

P9. The method of any one of embodiments P1-P8, wherein the disease or condition is selected from among an ulcer, wound infection, an ischemic condition, a metabolic condition, cellulitis, impetigo, athlete's foot, thrush, vaginitis, ringworm, dermatitis, periodontal disease, tooth decay, hypertension, neuropathy, a weakened immune system, HIV/AIDS, radiation exposure, cystic fibrosis, tuberculosis and a viral infection.

P10. The method of embodiment P9, wherein the disease or condition is a metabolic condition.

P11. The method of embodiment P10, wherein the metabolic condition is diabetes mellitus.

P12. The method of embodiment P19, wherein the disease or condition is an ischemic condition.

P13. The method of embodiment P12, wherein the ischemic condition is associated with 15atherosclerosis, peripheral vascular disease and/or venous insufficiency.

P14. The method of embodiment P9, wherein the disease or condition is a viral infection.

P15. The method of embodiment P14, wherein the virus is SARS-CoV-2 virus, herpes virus or human papillomavirus.

20P16. The method of embodiment P15, wherein the virus is herpes zoster virus or herpes simplex virus.

P17. The method of any one of embodiments P1-P16, wherein the one or more polypeptides of embodiments H1-H10 of the composition, of the pharmaceutical composition, or of the combination exhibit biofilm removal activity.

P18. The method of embodiment P17, wherein the biofilm associated with the disease or condition is reduced by at least 20%.

P19. The method of any one of embodiments P1-P18, wherein the one or more polypeptides of embodiments H1-H10 of the composition, of the pharmaceutical composition, or of the combination do not exhibit one or both of microbicidal activity and microbiostatic activity.

P20. The method of any one of embodiments P1-P18, wherein the one or more polypeptides of embodiments H1-H10 of the composition, of the pharmaceutical composition, or of the combination do not exhibit one or both of bactericidal activity and bacteriostatic activity.

P21. The method of any one of embodiments P1-P18, wherein the composition, pharmaceutical composition, or combination does not exhibit one or both of microbicidal activity and microbiostatic activity.

P22. The method of any one of embodiments P1-P18, wherein the composition, pharmaceutical composition, or combination does not exhibit one or both of bactericidal activity and bacteriostatic activity.

P23. The method of any one of embodiments P1-P20, wherein the composition, pharmaceutical composition, or combination comprises one or more of biofilm removal activity, microbiostatic, and/or microbicidal activity.

P24. The method of any one of embodiments P1-P20, wherein the composition, pharmaceutical composition, or combination comprises biofilm removal activity, and one or more of microbiostatic activity and microbicidal activity.

P25. The method of any one of embodiments P1-P20, wherein the composition, pharmaceutical composition, or combination comprises biofilm removal activity, and one or more of bacteriostatic activity and bactericidal activity.

P26. The method of any one of embodiments P1-P20, wherein the composition, pharmaceutical composition, or combination comprises biofilm removal activity

P27. The method of any one of embodiments P1-P20, wherein the composition, pharmaceutical composition, or combination consists essentially of biofilm removal activity.

P28. The method of any one of embodiments P1-P20, wherein the composition, pharmaceutical composition, or combination consists of biofilm removal activity.

P29. The method of any one of embodiments P1-P20and P23-P26, wherein there is biofilm associated with the disease or condition and the amount of viable microbes associated with the biofilm is reduced and/or the growth of microbes associated with the biofilm is reduced.

P30. The method of embodiment P29, wherein the microbes comprise one or more of bacteria, fungi and viruses.

P31. The method of embodiment P30, wherein the amount of one or more of viable bacteria, fungi and viruses associated with the biofilm is reduced and/or the growth of one or more of bacteria, fungi and viruses associated with the biofilm is reduced.

P32. The method of any one of embodiments P1-P20and P23-P26, wherein the composition, pharmaceutical composition, or combination comprises an antibiotic.

P33. The method of embodiment P32, wherein the one or more polypeptides of embodiments H1-H10 of the composition, of the pharmaceutical composition, or the combination and the antibiotic are administered serially, sequentially, intermittently, concurrently or simultaneously to the subject.

P34. The method of embodiment P32, wherein the one or more polypeptides of embodiments A1-A10 of the composition, of the pharmaceutical composition, or the combination is/are administered prior to the administration of the antibiotic.

P35. The method of any one of embodiments P1-P20 and P23-P26, wherein the composition, pharmaceutical composition, or combination comprises an antiseptic agent and/or an antimicrobial agent.

P36. The method of embodiment P35, wherein the one or more polypeptides of embodiments H1-H10 of the composition, of the pharmaceutical composition, or of the combination and the antiseptic agent and/or the antimicrobial agent are administered serially, sequentially, intermittently, concurrently or simultaneously to the subject.

P37. The method of embodiment P36, wherein the one or more polypeptides of embodiments H1-H10 of the composition, of the pharmaceutical composition, or the combination is/are administered prior to the administration of the antiseptic agent and/or 25the antimicrobial agent.

P38. The method of any one of embodiments P35-P37, wherein the antiseptic agent comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, a nucleoside analog, and plant extracts.

P39. The method of any one of embodiments P1-P38, wherein the composition, pharmaceutical composition, or combination is administered to the skin, keratin, hair, a tooth, a tissue or an internal organ of the subject.

P40. The method of embodiment P39, wherein the tissue is epithelial or connective tissue.

P41. The method of embodiment P39, wherein the tissue is a membrane.

P42. The method of embodiment P41, wherein the membrane is a cutaneous membrane, mucous membrane, serous membrane or synovial membrane.

P43. The method of any one of embodiments P40-P42, wherein the tissue is injured, 10damaged and/or infected.

P44. The method of embodiment P43, wherein the tissue injury, damage and/or infection comprises a sore, wound, burn, inflamed gingiva, a skin ulcer, or an ulcer in the stomach mucosa or intestinal mucosa.

P45. The method of embodiment P44, wherein the wound is a chronic wound.

Q1. A device, comprising the composition of any one of embodiments K1-K33, the pharmaceutical composition of any one of embodiments L1-L13 and L41-L47, the composition of any one of embodiments M1-M19 and M25-M27, or the combination of any one of embodiments N1-N26.

Q2. The device of embodiment Q1 that is an endoscope or catheter.

Q3. The device of embodiment Q1 that is a wound dressing, a topical patch, syringe, an inhaler, a dosage cup, a dropper, a pump, a spray bottle, an aerosol container or an applicator for administering the composition, pharmaceutical composition or combination.

Q4. The device of embodiment Q3 that is a pump for irrigation of a wound or sore with the composition, pharmaceutical composition or combination.

Q5. The device of embodiment Q3 that is a spray bottle or aerosol container for coating a wound or sore with the composition, pharmaceutical composition or combination.

R1. A kit, comprising (a) the composition of any one of embodiments K1-K33, the pharmaceutical composition of any one of embodiments L1-L13 and L41-L47, the composition of any one of embodiments M1-M19 and M25-M27, or the combination of any one of embodiments N1-N26 and (b) a device for administration of the composition, pharmaceutical composition or combination.

R2. The kit of embodiment R1, wherein the composition, pharmaceutical composition or combination is contained in the device for administration.

R3. The kit of embodiment R1, wherein the composition, pharmaceutical composition or combination is present as a separate component that is distinct from the device.

R4. The kit of any one of embodiments R1-R3, wherein the device is a dressing, a topical patch, a pump, a spray bottle, an aerosol container, a syringe, an inhaler, a dosage cup, a dropper, or an applicator.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology has been described with reference to specific implementations. The terms and expressions that have been utilized herein to describe the technology are descriptive and not necessarily limiting. The terms and expressions that have been employed are used as terms of description and not of limitation and use of such terms and expressions do not exclude any equivalents of the features shown and described or portions thereof, and various modifications are possible within the scope of the technology claimed. Thus, it should be understood that although the present technology has been specifically disclosed by representative embodiments and optional features, modification and variation of the concepts herein disclosed may be resorted to by those skilled in the art, and such modifications and variations are considered within the scope of this technology.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

We claim:

1. An isolated, recombinant or synthetically produced polypeptide comprising a consecutive sequence of amino acids that is 80% or more identical, or 90% or more identical to a corresponding consecutive sequence of amino acids set forth in SEQ ID NO:1.

2. The polypeptide of claim 1, comprising a sequence of amino acids that is 80% or more identical, or 90% or more identical to the sequence of amino acids set forth in SEQ ID NO: 1.

3. An isolated, recombinant, or synthetically produced polypeptide comprising a portion of a sequence of amino acids that is 80% or more identical, 90% or more identical, or 100% identical to the sequence of amino acids set forth in SEQ ID NO: 1, wherein the portion exhibits biofilm removal activity and/or enzyme activity.

4. The polypeptide of any one of claims 1-3, wherein the polypeptide exhibits enzyme activity.

5. The polypeptide of claim 4, wherein the polypeptide exhibits hydrolase activity.

6. The polypeptide of claim 4, wherein the polypeptide exhibits cutinase activity.

7. The polypeptide of any one of claims 4-6, wherein the enzyme activity is thermally stable.

8. The polypeptide of any one of claims 1-7, wherein the polypeptide exhibits biofilm removal activity, microbicidal activity, and/or microbiostatic activity.

9. The polypeptide of any one of claims 1-7, wherein the polypeptide exhibits biofilm removal activity, bactericidal activity and/or bacteriostatic activity.

10. The polypeptide of any one of claims 1-7, wherein the polypeptide exhibits biofilm removal activity but does not exhibit one or both of bactericidal activity and bacteriostatic activity.

11. An isolated, recombinant or synthetically produced polynucleotide encoding the polypeptide of any one of claims 1-10.

12. The polynucleotide of claim 11, wherein the polynucleotide encodes the amino acid sequence of SEQ ID NO:1.

13. The polynucleotide of claim 11, comprising a consecutive sequence of nucleotides that is 80% or more identical, or 90% or more identical to a corresponding consecutive sequence of nucleotides set forth in SEQ ID NO: 2 or SEQ ID NO: 3.

14. The polynucleotide of claim 11, comprising (i) a sequence of nucleotides that is 80% or more identical, or 90% or more identical to the sequence of nucleotides set forth in SEQ ID NO:2, (ii) a sequence of nucleotides that is 80% or more identical, or 90% or more identical to the sequence of nucleotides set forth in SEQ ID NO:3, (iii) the sequence of nucleotides set forth in SEQ ID NO:2, or (iv) the sequence of nucleotides set forth in SEQ ID NO:3.

15. The polynucleotide of any one of claims 11-14, wherein the polypeptide encoded by the polynucleotide exhibits enzyme activity.

16. The polynucleotide of claim 15, wherein the enzyme activity is thermally stable.

17. The polynucleotide of any one of claims 1-16, wherein the polypeptide encoded by the polynucleotide exhibits biofilm removal activity, microbicidal activity, and/or microbiostatic activity.

18. A vector, comprising the polynucleotide of any one of claims 11-17.

19. The vector of claim18 that is an expression vector.

20. The vector of claim 18 or claim 19, comprising the sequence of nucleotides set forth in SEQ ID NO:3.

21. A composition, comprising one or more polypeptides of claims 1-10.

22. The composition of claim 21, wherein the polypeptide(s) of claims 1-10 exhibits enzyme activity and the composition does not comprise any other polypeptide that exhibits enzyme activity.

23. The composition of claim 21 or claim 22, wherein the polypeptide(s) of claims 1-10 exhibits biofilm removal activity.

24. The composition of claim 23, wherein the amount of the one or more polypeptides of claims 1-10 is effective to reduce the amount of a biofilm by about 20% or more.

25. The composition of claim 21 or claim 22, further comprising an excipient selected from among a protectant, surfactant, buffer, and/or bulking agent.

26. The composition of any one of claims 21-25, wherein the composition is in the form of a liquid, solid, semi-solid or mixture of a liquid, solid and/or semi-solid.

27. The composition of any one of claims 21-26, wherein the composition is in the form of a cream, lotion, emulsion, oil, butter, paste, balm, stick, foam, gel, serum, ointment, mousse, powder, lyophilizate, semi-solid formulation, patch, spray or aerosol.

28. The composition of any one of claims 21-27, wherein the composition is formulated for administration to a human.

29. The composition of any one of claims 21-28, wherein the composition exhibits biofilm removal activity, microbicidal activity, and/or microbiostatic activity.

30. The composition of any one of claims 21-29, wherein the composition is a pharmaceutical composition.

31. The composition of claim 30, further comprising a pharmaceutically acceptable excipient.

32. The composition of any one of claims 21-31 for treating a disease or condition associated with a microbial infection and/or susceptible to microbial infection.

33. The composition of any one of claims 21-32, further comprising an additional therapeutically active agent.

34. The composition of claim 33, wherein the additional therapeutically active agent comprises one or more of an analgesic agent, an anti-inflammatory agent and an antimicrobial agent.

35. The composition of claim 33, wherein the additional therapeutically active agent comprises one or more of an antibacterial agent, an antifungal agent and an antiviral agent.

36. The composition of claim 33, wherein the additional therapeutically active agent 25comprises an antibiotic.

37. The composition of any one of claims 21-36, further comprising an antiseptic agent and/or antimicrobial agent.

38. The composition of claim 37, wherein the antiseptic agent comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, a nucleoside analog, and plant extracts.

39. The composition of any one of claims 21-38, further comprising one or more of bleach, chlorine compounds, an aldehyde and a detergent.

40. A combination, comprising:
(1) one or more polypeptides of claims 1-10, and
(2) an agent, wherein the agent is:
(a) a therapeutically active agent that does not comprise a polypeptide of claim 1-10, or
(b) a surface treatment agent that does not comprise a polypeptide of claims 1-10;
wherein (1) and (2) are each in a separate composition or are in the same composition.

41. The combination of claim 40, wherein the amount of (1) is effective to reduce the amount of a biofilm by about 20% or more.

42. The combination of claim 40 or claim 41, wherein (2) comprises one or more of: an analgesic agent, an anti-inflammatory agent, a disinfecting agent, a sanitizing agent, a cleaning agent, an antiseptic agent and an antimicrobial agent.

43. The combination of claim 40 or claim 41, wherein (2) comprises one or more of: a disinfecting agent, a sanitizing agent, a cleaning agent, an antiseptic agent, and an antimicrobial agent.

44. The combination of claim 43, wherein (2) comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, plant extracts, bleach, chlorine compounds, alcohol, an aldehyde and a detergent.

45. The combination of any one of claims 40-42, wherein (2) comprises an antimicrobial agent.

46. The combination of any one of claims 40-42, wherein (2) comprises one or more of an antibacterial agent, an antifungal agent and an antiviral agent.

47. The combination of claim 46, wherein (2) comprises a nucleoside analog and/or imidazole or derivative thereof.

48. The combination of any one of claims 40-42, wherein (2) comprises an antibiotic.

49. The combination of any one of claims 40-48, wherein the amount of (1) and (2) are such that when a surface is contacted with (1) and (2) the combination is effective to reduce the amount of one or more of viable bacteria, fungi and viruses, or reduce the growth of one or more of bacteria, fungi and viruses.

50. The combination of any one of claims 40-48, wherein the amount of (1) and (2) are such that when (1) and (2) are administered to a subject the combination is effective to reduce the amount of one or more of viable bacteria, fungi and viruses, or reduce the growth of one or more of bacteria, fungi and viruses in or on the subject.

51. The combination of any one of claims 40-48, wherein the amount of (1) and (2) are such that when a surface is contacted with (1) and (2) the combination is effective to reduce a greater amount of one or more of viable bacteria, fungi and viruses on the surface than the amount of reduction of the one or more of viable bacteria, fungi and viruses on the surface when it is contacted with only one of (1) or (2).

52. The combination of any one of claims 40-48, wherein the amount of (1) and (2) are such that when (1) and (2) are administered to a subject the combination is effective to reduce a greater amount of one or more of viable bacteria, fungi and viruses in or on the subject than the amount of reduction of the one or more of viable bacteria, fungi and viruses in or on the subject when only one of (1) or (2) is administered to the subject.

53. The combination of any one of claims 40-48, wherein (1) and (2) are administered to a subject, or contacted with a surface, serially, sequentially, intermittently, concurrently or simultaneously.

54. A method of removing biofilm and/or of treating a surface, comprising contacting the biofilm or surface with a composition of any one of claims 21-39 or a combination of any one of claims 40-53.

55. The method of claim 54, wherein the one or more polypeptides of claims 1-10 of the composition or combination exhibit biofilm removal activity and the polypeptide(s) is/are in an amount effective to reduce the amount of a biofilm associated with the surface by about 20% or more.

56. The method of claim 54 or claim 55, wherein treating comprises one or more of biofilm removal activity, microbiostatic, and/or microbicidal activity.

57. The method of claim 54 or claim 55, wherein treating comprises biofilm removal activity, and one or more of microbiostatic activity and microbicidal activity.

58. The method of any one of claims 54-57, wherein treating comprises reducing the amount of one or more of viable bacteria, fungi and viruses, and/or stopping the growth of one or more of bacteria, fungi and viruses.

59. The method of any one of claims 54-57, wherein treating comprises reducing the amount of one or more of viable SARS-CoV-2 virus, herpes virus and human papillomavirus or stopping the growth of one or more of viable SARS-CoV-2 virus, herpes virus and human papillomavirus.

60. The method of any one of claims 54-59, wherein treating comprises one or more of disinfection, antisepsis, sanitization and cleaning.

61. The method of any one of claims 54-60, wherein the surface is a hard surface, a soft surface or a porous surface.

62. The method of any one of claims 54-60, wherein the surface is a biological surface.

63. The method of claim 54 or claim 55, wherein removing biofilm further comprises reducing the amount of viable microbes and/or reducing the growth of microbes.

64. The method of any one of claims 54, 55 and 63, wherein the biofilm is in or on a 20biological substance.

65. The method of any one of claims 54-62 and 64, wherein the surface or biological substance is selected from among skin, keratin, hair, teeth, tissues and internal organs.

66. The method of claim 65, wherein the tissue is a cutaneous membrane, mucous membrane, serous membrane or synovial membrane.

67. The method of claim 66, wherein the tissue is injured or damaged.

68. The method of claim 67, wherein the tissue injury or damage comprises a sore, wound, burn, a skin ulcer, or an ulcer in the stomach mucosa or intestinal mucosa.

69. The method of claim 68, wherein the wound is a chronic wound.

70. The method of any one of claims 54-62 and 64-69, wherein the composition or combination comprises an antibiotic.

71. The method of claim 70, wherein the one or more polypeptides of claims 1-10 of the composition or combination and the antibiotic are contacted with the biofilm or surface serially, sequentially, intermittently, concurrently or simultaneously.

72. The method of any one of claims 54-62 and 64-69, wherein the composition or combination comprises an antiseptic agent and/or antimicrobial agent.

73. The method of claim 72, wherein the one or more polypeptides of claims 1-10 of the composition or combination and the antiseptic agent and/or antimicrobial agent are contacted with the biofilm or surface serially, sequentially, intermittently, concurrently or simultaneously.

74. The method of claim 72 or claim 73, wherein the antiseptic agent comprises one or more of an alcohol, a peroxide, permanganate, phenol or derivative thereof, an iodine-containing composition, a quinolone or derivative thereof, a quaternary ammonium compound, a biguanide, imidazole or derivative thereof, a hydroxypyridone, a nucleoside analog, and plant extracts.

75. The method of any one of claims 54-62 and 61, wherein the surface is an inanimate surface.

76. The method of any one of claims 54-61 and 63, wherein the biofilm is in or on an inanimate object.

77. The method of claim 75 or claim 76, wherein the composition or combination comprises one or more of a disinfecting agent, a sanitizing agent, a cleaning agent, and an antimicrobial agent.

78. The method of claim 77, wherein the one or more polypeptides of claims 1-10 of the composition or combination and the one or more of a disinfecting agent, a sanitizing agent, a cleaning agent, and an antimicrobial agent is/are contacted with the biofilm or 25surface serially, sequentially, intermittently, concurrently or simultaneously.

79. The method of claim 77 or claim 78, wherein the composition or combination comprises one or more of bleach, a chlorine compound, alcohol, peroxide, a phenol or derivative thereof, an aldehyde and a detergent.

80. The method of claim 79, wherein the one or more polypeptides of claims 1-10 of the 30composition, pharmaceutical composition, or combination and the one or more of bleach, a chlorine compound, alcohol, peroxide, a phenol or derivative thereof, an aldehyde and a detergent is/are contacted with the biofilm or surface serially, sequentially, intermittently, concurrently or simultaneously.

81. A method of treating a disease or condition of a subject comprising administering, to a subject in need thereof, a composition of any one of a composition of any one of claims 21-39 or a combination of any one of claims 40-53.

82. The method of claim 81, wherein there is biofilm associated with the disease or condition.

83. The method of claim 82 or claim 83, comprising administering a therapeutically effective amount of the composition or combination.

84. The method of any one of claims 81- 83, wherein the disease or condition is associated with a microbial infection and/or susceptible to microbial infection.

85. The method of any one of claims 81-84, wherein the disease or condition is associated with tissue injury or damage.

86. The method of claim 85, wherein the tissue injury or damage comprises a sore, wound or burn.

87. The method of claim 86, wherein the tissue injury or damage comprises an ulcer in the stomach mucosa or intestinal mucosa or a skin ulcer.

88. The method of any one of claims 81-87, wherein the disease or condition is selected from among an ulcer, wound infection, an ischemic condition, a metabolic condition, cellulitis, impetigo, athlete's foot, thrush, vaginitis, ringworm, dermatitis, periodontal disease, tooth decay, hypertension, neuropathy, a weakened immune system, HIV/AIDS, radiation exposure, cystic fibrosis, tuberculosis and a viral infection.

89. The method of claim 88, wherein the disease or condition is a metabolic condition.

90. The method of claim 89, wherein the metabolic condition is diabetes mellitus.

91. The method of claim 88, wherein the disease or condition is an ischemic condition.

92. The method of claim 91, wherein the ischemic condition is associated with atherosclerosis, peripheral vascular disease and/or venous insufficiency.

93. The method of claim 88, wherein the disease or condition is a viral infection.

94. The method of claim 93, wherein the virus is SARS-CoV-2 virus, herpes virus or human papillomavirus.

95. The method of any one of claims 81-94, wherein the one or more polypeptides of claims 1-10 of the composition or combination exhibit biofilm removal activity.

96. The method of claim 95, wherein the biofilm associated with the disease or condition is reduced by at least 20%.
